(19) United States
(12) Patent Application Publication
(10) Pub. No.: US 2024/0319212 A1
(43) Pub. Date: Sep. 26, 2024

(54) METHOD OF MEASURING THE ENDOCYTIC VITAMIN D STATUS

(71) Applicant: Immundiagnostik AG, Bensheim (DE)

(72) Inventors: Franz Paul Armbruster, Bobenheim-Roxheim (DE); Liliana Schaefer, Frankfurt (DE)

(73) Assignee: Immundiagnostik AG, Bensheim (DE)

(21) Appl. No.: 18/610,782

(22) Filed: Mar. 20, 2024

Related U.S. Application Data
(63) Continuation-in-part of application No. 16/958,850, filed on Jun. 29, 2020, now abandoned.

(30) Foreign Application Priority Data
Jan. 3, 2018 (DE) 10 2018 100 096.0

(51) Int. Cl. G01N 33/82; G01N 33/543

(52) U.S. Cl. CPC G01N 33/82; G01N 33/54306; G01N 2470/06

(57) ABSTRACT

Test kit, composition and a method of measuring vitamin D and its metabolites in a sample of bodily fluid from a subject, which sample contains vitamin D binding protein (DBP), comprising the steps of contacting said sample with megalin and/or a soluble fragment thereof under binding conditions to form a ternary complex containing DBP, a vitamin D metabolite and megalin or a fragment thereof; determining the amount of DBP bound by megalin; and relating the amount of megalin-bound DBP to the effective status of vitamin D in the circulation of said subject.

This application is a continuation-in-part of U.S. application Ser. No. 16/958,850, filed on Jun. 29, 2020, which is a 371 national stage of Application No. PCT/EP2019/050121, filed Jan. 3, 2019, which claims priority from German Patent Application No. DE 10 2018 100 096.0, filed on Jan. 3, 2018.

### REFERENCE TO A SEQUENCE LISTING SUBMITTED ELECTRONICALLY

The official copy of the sequence listing is submitted electronically via Patent Center as an XML formatted sequence listing with a file named IDK00126US02.xml, created on Mar. 20, 2024, and having a size of 13,000 bytes, and is filed concurrently with the specification. The sequence listing contained in this XML formatted document is part of the specification and is herein incorporated by reference in its entirety.

### FIELD OF THE INVENTION

The present application relates to assays involving biological materials of a specific nature and in particular to an assay for measuring the endocytable vitamin D concentration in a biological sample of a subject, notably blood or serum, and for detection or diagnosis of diseases or conditions related to vitamin D status in blood or serum (G01N 2333/00, G01N2800/00)

### BACKGROUND OF THE INVENTION

The metabolic pathway leading to the synthesis of active vitamin D involves three reactions that occur in different tissues. In humans the synthesis is initiated in the skin with a UV light-mediated cleavage to produce cholecalciferol (vitamin D₃, VD₃). The other vitamin D isomer “ergocalciferol” (vitamin D₂, VD₂) occurs in plants and is taken up with the food. Both vitamin D isomers are metabolized in the liver to 25-hydroxyvitamin D [25(OH)D] which is also the major circulatory form (prohormone). This second step is catalyzed by a cytochrome P450 enzyme, a NAPH-hemoprotein reductase, while the identity of the hepatic 25-hydroxylase still requires more elucidation. The 25(OH)D then becomes 1α-hydroxylated in the kidney to 1α,25-dihydroxyvitamin D or calcitriol which is the physiologically active form (D-hormone). Calcitriol regulates the absorption of calcium in the intestines, the mineralization of the bones, the differentiation of osteoblasts, the synthesis of bone matrix and neuromuscular functions. It is common medical knowledge that 25(OH)D levels in serum lower than 15 ng per mL serum (37.5 nmol/L) cause a rise of the parathyroid hormone level and leads to an increase in bone resorption (Chapuy M C et al. in J Clin Endocrinol Metab 1996; 81:1129-33). A vitamin D deficiency may be caused by gastro-intestinal diseases, liver dysfunction, malabsorption drug-induced heightened metabolism, genetic defects or insufficient exposure to sunlight. Vitamin D deficiency is a known risk factor for senile osteoporosis. Generally, a deficiency of less than 5 ng 25(OH)D per mL serum (12.5 nmol/L) is regarded severe, causing rickets in children and osteomalacia in adults (Scharla et al. Exp Clin Endocrinol. Diabetes, 1996, 104:289-292). Excess of vitamin D due to overdosing causes hypercalcemia. It is not clear whether there is a definitive difference between the effects of VD₂ and VD₃ or whether they are equally effective in the raising of serum 25(OH)D, particularly at lower doses of vitamin D (cf: Tripkovic L et al, Am J Clin Nutr. 2012 June; 95(6): 1357-1364; Am J Clin Nutr. 2017 August; 106(2):481-490).

Vitamin D binding protein (DBP) is a member of the albuminoid superfamily. The 56-58 kDa glucoprotein can bind vitamin D metabolites as well as fatty acids and other endotoxins. It is thought that DBP acts as a reservoir in situations of deficiency, increasing the half-life of vitamin D, but also protects against vitamin D intoxication. The concentration of DBP in blood is stably maintained within a relatively narrow range (323-460 mg/L (5.52-7.93 mol/L)) in normal subjects, even in pathological conditions and disorders of the calcium metabolism. Except in pregnancy, no high DBP concentrations have been observed. DBP in serum can be measured by numerous methods, including immunoturbidimetry (Hamashima et al, Clinica Chimica Acta 321 (2002) 23-28). DBP is known to produce chemokinetic effects on neutrophil granulocytes, activate macrophages and sequester actin upon tissue damage.

The DBP level in serum is about 20-fold higher than of vitamin D and therefore 2 to 5% only will be occupied by vitamin D metabolites. For measurement, the vitamin D metabolites are released from DBP by enzymatic digestion, denaturing and/or ligand displacement (EP 2 126 586 B1, WO 99/67211; EP 0 753 743, WO 2004/063704). The releasing agents include all types of detergents and surfactants (EP 2 955 516 B1) as well as structural analogs of vitamin D such as warfarin, salicylic compounds, certain sulfonic acids, toluene sulfonic acids, naphthalene sulfonic acid, anilinonaphthalene sulfonic acids, etc. (WO 03/023391). The release of vitamin D from DBP is a decisive step in most laboratory procedures, in particular as the vitamin D metabolites are hydrophobic and cholesterol-like, bound by numerous serum proteins, and so their quantitative determination is technically difficult and results open to interpretation. The confusion as to the vitamin D status gave rise to collaborative initiative led by the Office of Dietary Supplements of the U.S. National institutes of Health and there have recently been proposals for assessing the vitamin D status via a determination of the “free 25(OH)D” in serum, say the 25(OH)D not bound by DBP or albumin (for review: The Importance of 25-Hydroxyvitamin D Assay Standardization and the Vitamin D Standardization Program. J AOAC Int. 2017, 100(5): 1223-1224; and Malmstroem S et al, J AOAC Int. 2017, 100(5): 1323-7).

Calcium and phosphorus are essential minerals required for many critical biologic functions including cell signaling, energy metabolism, skeletal growth and integrity. Calcium and phosphate homeostasis are maintained primarily by regulation of epithelial calcium and phosphate cotransport in the kidney and intestine, processes that are tightly regulated by hormones including calcitriol, fibroblast growth factor 23 (FGF23) and parathyroid hormone (PTH). In patients with chronic kidney disease (CKD), as renal function declines, disruption of feedback loops between these hormones have adverse consequences on several organ systems, including the skeleton, heart, and vascular system. Complications include vascular calcification, stroke, skeletal fracture, and increased risk of death. Increased FGF23 and PTH concentrations, and vitamin D deficiency contributes to the pathogenesis. Therefore, treatment of patients is focused on restoring the feedback loops to maintain normal calcium and phosphate balance to prevent skeletal and cardiovascular complications. Recent evidence is further linking the vitamin D status to disorders such as cancer, diabetes, depression, etc. leading to a higher demand of routine testing for vitamin D levels even in healthy patients. The importance of the vitamin D status in research and health cannot therefore be overstated and clinical laboratories are confronted with the challenge of increasing test numbers and the need to identify persons truly suffering from a low vitamin D status. The state of the art represents a problem.

### SUMMARY OF THE INVENTION

The application provides an assay and a method of measuring the effective vitamin D level (including the vitamin D metabolites of the storage form) in a sample of bodily fluid in the presence of vitamin D binding protein (DBP), comprising the steps of a) contacting said sample with megalin/LRP2 (low-density lipoprotein related protein 2) and/or a soluble fragment thereof under binding conditions to form a complex containing DBP, vitamin D or a metabolite thereof and megalin/LRP2 or a fragment thereof; b) determining the amount of DBP:vitamin D and/or any one of its components; and c) relating the amount of megalin-bound complex of DBP:vitamin D to the effective vitamin D status in said subject.

In a preferred embodiment, the assay and method comprise the use of a fragment of megalin which binds none of the other ligands of megalin/LRP2, and/or a fusion protein with said fragment of megalin which can bind the DBP:VD complex. A preferred embodiment of said method comprises a differential measurement of hydroxylated cholecalciferol (25-hydroxyvitamin D₃) in the presence of hydroxylated ergocalciferol (25-hydroxyvitamin D₂), 24,25-dihydroxyvitamin D₂, and/or 24,25-dihydroxyvitamin D₃.

The disclosure encompasses the use of the endogenous DBP present in serum but may include the addition of DBP to obtain a standard concentration of DBP in the test samples. The disclosure may further encompass contacting additionally said sample with cubilin and/or a soluble fragment thereof as cubilin is known to facilitate the endocytic process (Nykjaer A, et al Cubilin dysfunction causes abnormal metabolism of the steroid hormone 25(OH) vitamin D₃. PNAS U.S.A. (2001) 98(24): 13895-900 [PUBMED: 11717447].

In one embodiment, the amount of DBP: VD bound by megalin is determined by turbidimetry or nephelometry or, notably, by an immunoassay for DBP. In another embodiment, the megalin and/or said soluble fragment thereof may be bound to particles or beads having diameters ranging from 50 to 200 nm. Alternatively, the disclosure teaches for the sake of completion an immunoassay selected from the group ELISA (enzyme-linked immunosorbent assay), RIA (radioimmunoassay), FIA (fluorescence immunoassay), LIA (luminescence immunoassay), or ILMA.

In another embodiment the method may comprise the steps of (a) providing a defined amount of megalin and/or a fragment thereof coupled to a solid phase; (b) contacting the sample with a solid phase; (c) creating conditions to allow binding of megalin and/or a fragment thereof to the complex formed between DBP and vitamin D metabolite, wherein DBP alone is not bound by megalin and/or a fragment thereof; and washing the solid phase; (e) providing an antibody recognizing the complex of DBP:VD or, facultatively, megalin or a soluble fragment thereof; (f) contacting said megalin and/or fragment thereof bound to DBP:VD with an anti-DBP-antibody, and optionally, immobilizing the complex on the solid phase; and (g) determining the amount of antibody bound to the solid phase, and quantitating the vitamin D status in plasma or serum by correlation with a reference.

The disclosure also provides a test kit which comprises an antibody specific for DBP and a binding partner comprising megalin or a fragment thereof. The antibody may be one binding to DBP or vitamin D metabolite in said complex. In another embodiment, the test kit may comprise nanoparticles having bound megalin and/or soluble fragments thereof.

The stated object is achieved because the disclosure provides a method for direct quantitative determination of the effective vitamin D status in serum or plasma. The status is based on the major circulating vitamin D metabolite which is ready for endocytosis and internalization into cells of the kidney, or into cells having an endocytic megalin transport pathway, so that the prohormone (25(OH)D) will be 1α-hydroxylated to the physiologically active D-hormone (calcitriol). As ergocalciferol and cholecalciferol are both bound by DBP but the complex of DBP:VD₃ is predominantly bound by megalin, the disclosed method provides a status of prohormone capable of becoming the active hormone. Vitamin D metabolites such as 24,25(dihydroxy)vitamin-D₃, while they can still form a complex with DBP, are either much less bound by megalin or their concentration is too low in the circulation to impact the determined effective vitamin D status.

The DBP:VD₃ complex only is endocytosed by the megalin cubilin pathway into cells where the P450 1α-hydroxylase (Cyp27B1) is located on the outside of the mitochondrial membrane. Consequently, other vitamin D metabolites will either not become activated or their concentration in the circulation is too low for being relevant with respect to the measured vitamin D status. This applies in particular to 25-hydroxylated ergocalciferol [25(OH)D₂] which complex with DBP is not or much less bound by megalin (see FIG. 8B). The present disclosure therefore contradicts the free hormone hypothesis according to which only the non-protein-bound fraction (the free fraction) of vitamin D metabolites can enter cells and exert biologic effects.

The determination of the effective vitamin D status can be done in aqueous solution despite of the lipophilic nature of the vitamin D metabolites. There is no need for releasing the vitamin D metabolites from their binding partners (DBP, albumin, proteins of the albuminoid superfamily, etc.) as the method is based on the measurement of the complex of DBP:VD which will be bound or discriminated by megalin or a soluble fragment thereof. A reliable discrimination of vitamin binding protein having bound vitamin D is provided. Megalin or fragments thereof are used to discern the vitamin D status based on the endocytic DBP:VD complex which we submit represents the solely bioactivatable prohormone in the circulation.

Soluble megalin can be produced in mammalian cells by recombinant methods and purified by affinity chromatography. Megalin fragments soluble in aqueous solution (serum, plasma) are preferred as they allow conditions for the formation of DBP:VD complex close to physiological.

As disclosed, there is no need to release vitamin D from its binding partners as by prior art methods. Thus, there is no need for halogenated solvents, tensids and surfactants (PFOA, CTAB) which makes the method environmentally friendly. The lack of preanalytical purification and preparation steps further allows a direct determination of the effective vitamin D status immediately after sample collection. The concept of determining the formation of said megalin-bound complex can easily be adapted to platforms such as ELISA, turbidimetry and nephelometry. The proteinaceous components of said complex can be reliably quantified.

The present disclosure is further in conformity with clinical reports that 25-hydroxyvitamin D₃ more effective than 25-hydroxyvitamin D₂. The disclosure provides an effective status on basis of the endocytable prohormone which will be available for activation. While the 25-hydroxylated vitamin D₂ and D₃ isomers are equally bound by the DBP the provided method makes use of the different binding affinity of megalin to DBP when in interaction with 25-hydroxyvitamin D₃. The different binding affinity is striking. It is submitted that a fundamental biological mechanism has been discovered which physiological relevance cannot be overlooked. The present method allows therefore a discrimination of the activatable circulatory vitamin D metabolites and therefore provides a status of the readily activatable vitamin D metabolite (prohormone). With the information provided, vitamin D supplementation therapies can be adapted to the true physiological status of the subject, avoiding toxicity or inefficient therapies.

The principles of invention will now be described by reference to its advantages, representative examples and drawings which shall, however, not limit the gist of the invention which can be derived from the disclosure contained in the claims.

### BRIEF DESCRIPTION OF THE DRAWINGS

In the accompanying drawings,

FIGS. 1A-C are schematic representations showing (A) potential binding sites of human DBP (dimer) to megalin as predicted by PepSite 2 (Trabuco L G et al, PepSite: prediction of peptide-binding sites from protein surfaces in Nucleic Acids Res. 2012; 40(Web Server issue): W423-426); (B) the interaction between megalin/LRP2, cubilin and their known ligands on the outside of the luminal plasma membrane; and (C) the pathways from vitamin D via the prohormone (25(OH)D to the active hormone (1,25(OH)₂D-calcitriol) as well as excretion pathways;

FIGS. 2A-C are schematic representations of (A) chosen megalin fragments tested for binding of vitamin D binding protein: Cons M1: M1-K386 (signal peptide+A1-7+EGF-like ½); M2: (M1-25 signal peptide+E1024-K1429); M3: (M1-25 signal peptide+E2698-R3192; M4: (M1-25 signal peptide+P3510-A4048—not further examined); (B) the cDNA constructs of megalin fragments (M1, M2, and M3) cloned into expression vector pcDNA3 (Invitrogen, San Diego, CA); and (C) Western blots of secreted megalin fragments (M1-M3 dimers) and M2 monomer: HEK cell lysates and after Ni-NTA affinity purification from culture supernatants;

FIGS. 3A,B show Western blots of (A) megalin fragments from transfected HEK cell lysates (M1, M2, M3+control/Non; ˜20 μg) and (B) co-immunoprecipitations of the megalin-bound (M1, M2, M3) DBP (0.5 μg) in the presence or absence of VD₃ (0.2 μg) using anti-His-antibody (WB staining: anti-DBP Ab);

FIGS. 4A,B show Western blots (A) of Ni-NTA purified megalin fragments (M1, M2 and M3+Anti-IgG AB as control; ˜3 μg) and (B) co-immunoprecipitations of the complex of megalin bound DBP (2 μg) in the presence or absence of VD₃ (0.5 μg) using an anti-His-antibody (WB staining: anti-DBP Ab);

FIGS. 5A,B show (A) microscale thermophoresis analyses of megalin fragments M1, M2 and M3 and DBP (50 nM) in the presence or absence of VD₃ (50 nM); and (B) the effect of the VD₃ concentration on the affinity analysis;

FIGS. 6A,B show Western blots (A) of co-immunoprecipitated DBP from different human sera or plasma using purified megalin fragment M1 (3 μg); and a bar diagram (B) showing the results of an ELISA for total DBP and formed DBP/VD₃ bound by surface-coated megalin fragment (M1 or M2);

FIGS. 7A-C are graphs (A) showing the effect of added purified DBP on the formation of M2-megalin-bound DBP:25(OH)D₃ in a sample of human serum of a subject (RMS); and (B) the correlation between 25(OH)D₃ serum levels and the binding of DBP:25(OH)D₃ to megalin M2 fragment in human serum; and (C) the correlation between 25(OH)D₃ level and ternary complex DBP/25(OH)D₃/M2 and the same for the ternary complexes with 25(OH)D₂ and 24,25(OH)VD, respectively;

FIG. 8A,B show (A) microscale thermophoresis analyses of DBP and various 25(OH)D metabolites: (25(OH)D₃, 25(OH)D₂, 24,25(OH)D; and (B) microscale thermophoresis analyses of DBP:VD-metabolite when bound by megalin M2 fragment;

FIG. 9A,B are graphs showing the linearity range of megalin-bound DBP:25(OH)D₃ and in the presence of varying concentrations of 24,25(OH)VD and 25(OH)VD₂ at high concentrations (up to 50 ng/ml)

FIG. 10A,B are graphs showing the linearity range of megalin-bound DBP:25(OH)D₃ and in the presence of varying concentrations of 24,25(OH)VD and 25(OH)VD₂ for low concentrations (up to 19 ng/ml).

FIG. 11 shows a comparative diagram illustrating the results of microscale thermophoretic analysis of polyclonal anti-TH17-antibodies, raised against a 17 amino acid peptide of SEQ ID NO. 11. The analysis focuses on the different binding affinities (dissociation constants Kd) of these antibodies with two entities: megalin-bound DBP:25(OH)D₃ complex and apoprotein DBP alone.

### DETAILED DESCRIPTION OF THE INVENTION

The instant description provides a method of determining the amount of biologically effective vitamin D in a sample of bodily fluid from a subject. The method comprises the steps of contacting said sample with DBP and megalin or a functional fragment thereof under binding conditions to form a complex of DBP:vitamin D (or a metabolite of vitamin D) which will be specifically bound by megalin, or a functional fragment thereof, to form a ternary complex comprising DBP:vitamin D:megalin. The amount of formed ternary complex in said sample of bodily fluid can then be correlated to the biologically effective amount of vitamin D in said sample of bodily fluid.

In this context, the term “biologically effective vitamin D” or “effective vitamin D” defines and comprises all structural vitamin D molecules which form a complex with DBP that is recognized and bound by megalin or a functional fragment thereof to form a ternary complex. Those structural vitamin D molecules therefore comprise not only vitamin D (chole- and ergocalciferol) but also the 25-hydroxylated vitamin D metabolites, including the respective epimers. For many years, emphasis has been on measuring total levels of 25-hydroxyvitamin D [25(OH)D]. As the measured values were not consistent with physiologies there has recently been hypothesized that “free 25(OH)D” is a potentially better marker of the vitamin D status. The proposed assessment of “free 25(OH)D” however relies on calculations using levels of total 25(OH)D, albumin, and DBP and on the assumption of a constant affinity of DBP for the vitamin D metabolites. This hypothesis works with the assumption that the lipophilic vitamin D metabolites can passively diffuse through cell membranes and that serum DBP and albumin would decrease the readily available amount of vitamin D and the vitamin D status. The determinataion of the “free DBP-unbound fraction of 25(OH)D” shall give the “actual free and available vitamin D status.”

The vitamin D status is not only essential for normal kidney function and bone health but can also be linked to cancer development and some autoimmune diseases. Given the impact of the vitamin D status on human health, reliable methods are required for clinical practice.

The concentration of total “25-hydroxyvitamin D” in serum is mostly used in clinical practice but the discussion on the amount or proportion of “free or available” or hidden 25-hydroxy vitamin D” cannot be ignored. However, this hypothesis does not consider that epithelial cells and cells of other tissues (renal tubules, parathyroid gland, placenta etc.) have an endocytic pathways which enable the endocytic internalization of DBP-bound vitamin D metabolites. 25(OH)D is the key form of the prohormone for uptake and innercellular conversion to calcitriol. The vitamin D metabolites are mainly transported and bound in the circulation by the vitamin D binding protein. With prior art measurement methods, the amount of vitamin D metabolites that can become activated is not known and even less known is the amount of prohormone available for endocytic internalization and 1α-hydroxylation to the active D-hormone.

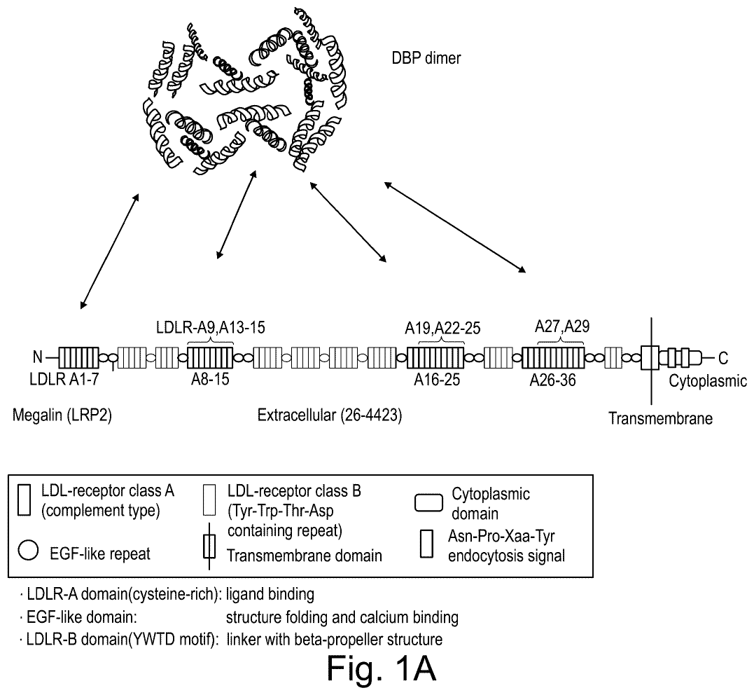

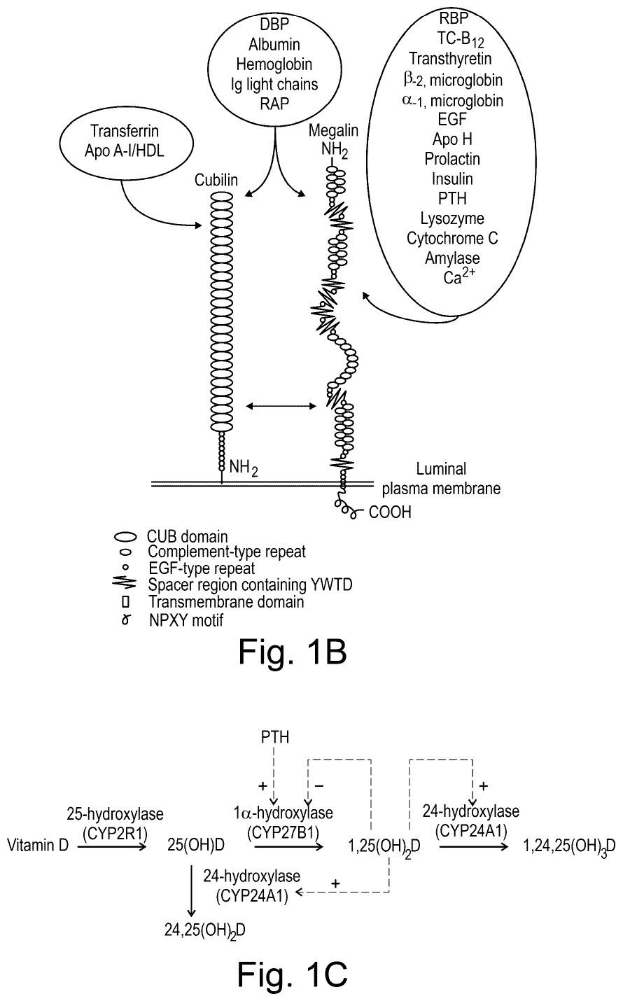

The present method makes use of that a ternary complex of vitamin D, DBP and megalin must form before the vitamin D metabolite can become internalized by endocytosis, either directly or following interaction with cubilin (cf. FIG. 1A-C). It is well established that vitamin D (cholecalciferol or ergocalciferol) is first hydroxylated in its 25-position to the prohormone in the liver and then present in the circulation. It also well established that the active D-hormone [1,25(OH)₂D] is synthesized by the enzyme 1α-hydroxylase (Cyp27B1-cytochrome P450) which is located within cells on the outside of the mitochondrial membrane (cf FIG. 1C). The 25-hydroxylated vitamin D metabolite must be first transported from the liver to the respective cells in the kidney. The kidney is the major source of calcitriol (1,25(OH)₂D) but also extrarenal cells, including lymphocytes, macrophages, keratinocytes, and cells of the parathyroid gland and pancreas can generate calcitriol.

It is known that 25-hydroxyvitamin D₃ is 300% more effective than 25-hydroxyvitamin D₂. In line therewith, the present disclosure provides a discrimination of endocytable DBP, say DBP having bound a vitamin D₃ metabolite. Megalin or fragments thereof are used to discern the physiologically activatable vitamin D₃ metabolite. Recombinant megalin fragments can be produced in mammalian cell lines and purified by affinity chromatography. A direct and fast determination of a vitamin D status is provided and there is no need for any additional pre-treatment or sample preparation. The assay's time-scale is, thus, reduced while providing physiological accurate and valid readings. The disclosed approach can easily be adapted to different platforms such as ELISA, turbidimetry and nephelometry.

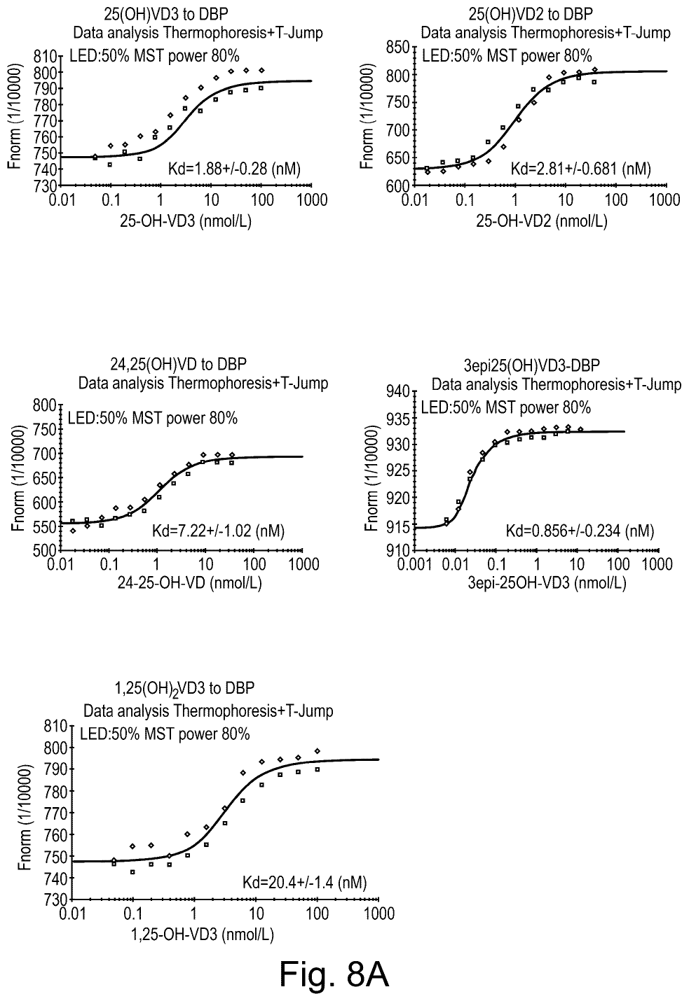

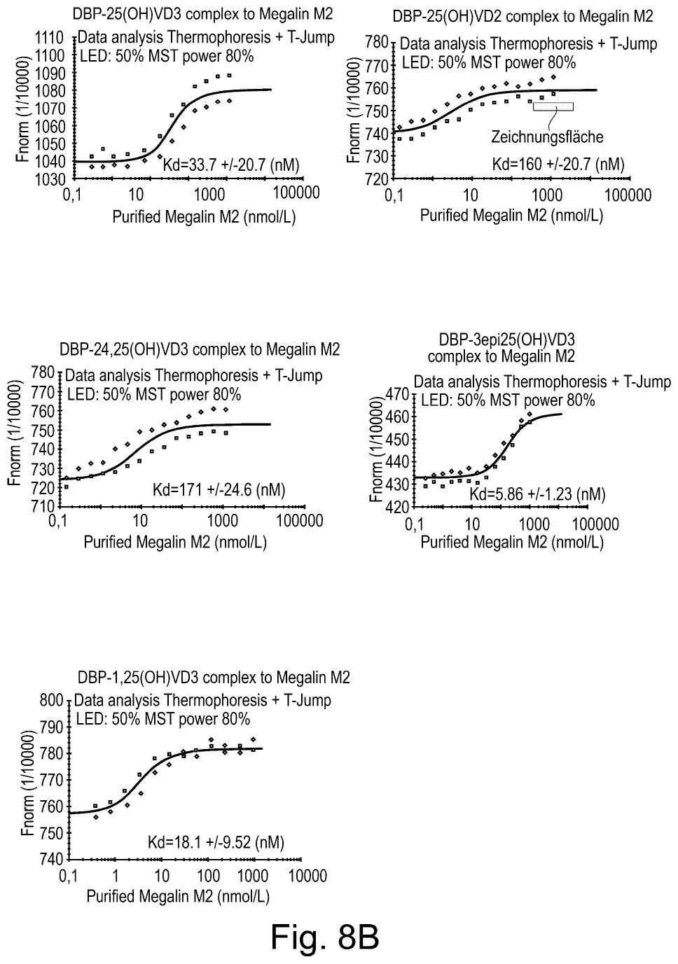

The novel vitamin D status corresponds to the physiologically activated, endocytable vitamin D, in particular, to the 25-hydroxyvitamin D₃ in serum or plasma. The concentrations of the other endocytable vitamin D metabolites are much lower. While the 25-hydroxylated vitamin D₂ and D₃ isomers are equally bound by DBP (FIG. 8A), the higher binding affinity of megalin to DBP:25(OH)VD₃ compared to DBP:25(OH)VD₂ and 24,25-hydroxyvitamin D is striking. A special role seems to be taken by the C3-epimer of 25-hydroxyvitamin D₃ [3-epi-25(OH)D₃], for which there is a near-total lack of data regarding its clinical significance. Although little is known regarding the in vivo importance of 3-epi-25(OH)D₃, clinical laboratories face the decision of whether or not to include 3-epi-25(OH)D₃ in the measurement of the vitamin D status. The data in FIGS. 8A,B show that the described method can adequately detect and resolve the C-3 epimeric form of 25(OH)D₃. Therefore, the present method allows a distinction for determining the most potent vitamin D metabolites and, in turn, provides a more accurate and physiologically valid vitamin D status which does not include the less active forms. With the information provided by the present disclosure, vitamin D supplementation therapies can therefore be adapted to the physiological status of the subject, avoiding toxicity or inefficient therapies.

In a preferred embodiment, the method comprises the use of a soluble fragment of megalin and/or a fusion of said soluble megalin fragment which binds the complex of DBP:VD metabolite but none or less of the numerous other ligands of LPR2/megalin. More precisely, a soluble fragment of megalin which has no affinity for albumin or anti-DBP antibody (no Ab cross-reactivity!). Said embodiment may comprise a surface-bound DBP-binding megalin fragment or a fusion protein thereof which contains an epitope comprising:

SEQ ID NO: 01
-D-N-G-N-C-I-H-R-A-W-L-C-D-R-D
or
SEQ ID NO: 2
-G-C-T-H-E-C-V-Q-E-P-F-G-A-K-C-

or both epitopes.

The sequence for affinity binding of human DBP:VD complex seems to contain:

SEQ ID NO: 03
-C-V-Q-E-P-
or
SEQ ID NO: 04
-I-H-R-A-W-

Other useful DBP binding epitopes within the megalin M2 region (E1024-K1429) are

(C19 sequence)
SEQ ID NO: 05
-S-D-F-N-G-G-C-T-H-E-C-V-Q-E-P-
(D5 sequence)
SEQ ID NO: 06
-C-Y-N-M-R-G-S-F-R-C-S-C-D-T-G
(A2 sequence)
SEQ ID NO: 07
-F-S-F-P-C-K-N-G-R-C-V-P-N-Y-Y

as determined by the interaction of DBP with megalin M2 (E1024-K1429)-SEQ ID NO: 09-using CelluSpots™ peptide array.

Preferred embodiment may comprise a determining of the megalin-bound complex of DBP:VD metabolites. This may be done using an antibody recognizing the complex of DBP:VD or after isolation of DBP, vitamin D or its metabolites or megalin or the soluble fragment thereof. In one aspect of the disclosure, the method may comprise providing mixtures of vitamin D metabolites and DBP for establishing standard samples.

In a preferred embodiment, the disclosure may comprise contacting said sample with an amount of added DBP to create standard conditions or a constant excess of DBP for a binding of the prohormone. The disclosure may further encompass contacting the sample with cubilin and/or a soluble fragment thereof.

Said embodiment may comprise a surface-bound DBP-binding megalin fragment or a fusion protein thereof which contains an epitope having any one or more of the amino acid sequence SEQ ID NO: 01 through SEQ ID NO: 10.

In one embodiment, the method of the disclosure may relate to a turbidimetric or nephelometric immunoassay. Said megalin and/or a soluble fragment thereof may be bound to nanoparticles having diameters ranging from 50 to 200 nm, so that the complex of DBP:VD is bound to said nanoparticles. The disclosure also provides an immunoassay selected from the group ELISA (enzyme-linked immunosorbent assay), RIA (radioimmunoassay), FIA (fluorescence immunoassay), L1A (luminescence immunoassay), or ILMA.

Consequently, the method may comprise the steps of (a) providing a defined amount of megalin and/or a soluble fragment thereof coupled to a solid phase; (b) contacting the sample with the solid phase having coupled megalin and/or a soluble fragment thereof; (c) creating conditions to allow binding of the complex formed by DBP and vitamin D metabolite, wherein DBP alone does not bind to megalin and/or a soluble fragment thereof; and washing the solid phase; (e) providing an antibody recognizing the ternary complex comprising DBP, vitamin D or its metabolite; (f) contacting said ternary complex with an antibody, optionally an antibody against DBP, and immobilizing the immunocomplex on the solid phase; and (g) determining the amount of antibody bound to the solid phase, and correlating the amount of bound antibody to the endocytic or activatable vitamin D status in blood, plasma, or serum by reference with a standard.

The instant disclosure further comprises a test kit for use in a method of measuring the effective vitamin D status which comprises an antibody specific for the ternary complex, megalin or a fragment thereof or DBP, or vitamin D and its metabolites. The disclosure also relates to a test kit for use in a method of measuring vitamin D metabolites in a sample of bodily fluid, comprising nanoparticles having bound megalin and/or soluble fragments thereof.

The achieved object is a simple and reliable method for a direct quantitative determination of the effective status of vitamin D and its metabolites in a sample. When the bodily fluid is blood, serum or plasma, the status describes the endocytable fraction of vitamin D metabolites in the circulation, say the fraction which can be processed intracellularly to the active hormone by respective target cells and tissues as needed. The novel vitamin D status can be determined in aqueous solution despite the highly lipophilic nature of vitamin D.

Vitamin D₃ is absorbed and processed by the organism easily and considered more potent than vitamin D₂ which has a shorter half-life and binds with less affinity to the vitamin D receptor (VDR). 1α,25(OH)D₃ and parathyroid hormone (PTH) influence the vitamin D metabolism by positive or negative regulation of the activity of the 1α-hydroxylase and 24-hydroxylase (see FIG. 1C). Again, vitamin D is 25-hydroxylated in the liver to 25(OH)D₃ or 25(OH)D₂, and further hydroxylated in kidney cells to its biologically active form 1α,25(OH)D. 24,25(OH)D is a further metabolite of 25(OH)D₃, but inactive and destined for excretion. In the circulation, vitamin D metabolites are tightly bound to DBP. Smaller amounts are bound to albumin and lipoproteins. The affinity of 25(OH)D (Ka=6×10⁵ M⁻¹) and 1,25(OH)₂D (Ka=5.4×10⁴ M⁻¹) for albumin is substantially lower than the affinity for DBP (25(OH)D (Ka=7×10⁸ M⁻¹) and 1,25(OH)₂D (Ka=4×10⁷ M⁻¹)). Because of the abundance of albumin in serum (650 μM) compared to DBP (5 μM), some vitamin D will likely be bound by albumin while not effectively available for endocytosis. Additionally, the vast majority of the DBP in serum is not occupied by any vitamin D metabolite. There has been no disclosure in the prior art defining a parameter which addresses the bioavailability of the vitamin D metabolites based on their binding to DBP and the following endocytic pathway. As DBP is present in high concentrations in the circulation it was so far not considered in the assessment of the vitamin D status. In the prior art, either total (bound and free) or free circulating vitamin D, separately, were considered sources of information for establishing a vitamin D status.

Megalin (also known as Low Density Lipoprotein receptor-related Protein 2, LRP2) is a multiligand binding cell receptor with structural similarities to the LDL receptor (LDLR). Megalin can be found in numerous cells and tissues, notably in the plasma membrane of absorptive epithelial cells (Farquhar M G et al, Soc. Nephrol. 6 (1): 35-47). LRP2/megalin is known to mediate the endocytosis of its ligands and can form complexes with cubilin which cubilin:megalin complexes are again able to (re)absorb molecules. The cubilin:megalin complex is inter alia responsible for the cellular uptake of lipids, VLDL, certain proteins (albumin, lactoferrin), cobalamin (vitamin B₁₂), and calcidiol. The instant disclosure proposes determining the amount of DBP:VD which is bound by megalin. This can be done for example by an ELISA against human DBP after “isolation or separation” of said ternary complex. This is preferred and the ELISA is already commercially available (Immundiagnostik AG, Bensheim). The term “effective vitamin D” describes formed complex of DBP:VD which has been bound by megalin (or a fragment thereof). The complex with megalin is considered to interact with cubilin so that the “activated vitamin D metabolite” will be endocytosed and subsequently 1α-hydroxylated to become the active hormone.

The effective vitamin D status represents an improvement over the prior art since it offers physiological information and a reading of the concentration of circulating vitamin D that will be processed to the D hormone. This status is different to the major circulating storage form because less active forms such as 25(OH)D₂ will not or much less contribute to the effective vitamin D status. The present disclosure provides a vitamin D status based on the amount DBP:VD, selectively and discriminably bound by megalin and/or cubilin. This endocytic complex therefore corresponds to the status of the effective vitamin D in serum or plasma. The term “bioavailable” as used in the prior art however refers to “total free” vitamin D which is speculative and is based on an assumed diffusion across the plasma membrane of cells.

There are contradictory views in the prior art with respect to the role of DBP. On the one hand, binding of vitamin D to DBP has been regarded a protection mechanism against excessive amounts of the free vitamin. This view is followed by groups supporting the “free hormone hypothesis”. On the other hand, studies in DBP knockout mice showed that these animals have significantly reduced plasma levels of 25(OH)D₃ and 1,25-(OH)₂D₃. On a vitamin D depleted diet, the animals suffered from vitamin D deficiency and bone formation defects. These results point to the development of alternative pathways which will not be relevant when DBP is present in the circulation.

Steroid hormones and sterols such as vitamin D are lipophilic and commonly require a carrier protein for effective delivery. There are many ligand-specific serum carriers of steroid hormones and sterols including corticosteroid-binding globulin (CBG) (glucocorticoids, mineralocorticoids), vitamin A (retinol)-binding protein, vitamin D-binding protein (DBP), sex hormone-binding globulin (SHBG) (estrogens, androgens), and thyroid hormone-binding globulin. For example, CBG and SHBG not only act as high affinity serum transporters, but also able bind to cell membranes in their ligand forms, suggesting alternative actions as signal transducers. In a similar fashion, DBP is a macrophage-activating factor (MAF) and actin-binder, which functions seem independent from the binding to vitamin D metabolites. The mechanisms by which ligands are released from binding globulins and acquired by target cells are crucial to steroid hormone signaling pathways.

This is particularly important for vitamin D where there is increasing evidence for extra-renal, intracrine, conversion of the pro-hormone (25(OH)D) to active D-hormone. The impact of vitamin D is then very much dependent on tissue-specific expression of the 1α-hydroxylase and the receptor for 1,25(OH)₂D, the nuclear vitamin D receptor (VDR). It has been estimated that concentrations of free 1,25(OH)₂D in serum are approximately 10⁻¹³ M, which is much less than the concentrations quoted for binding to the vitamin D receptor (dissociation constant (Kd)=approximately 10⁻¹⁰ M). Due to the obvious disparity between the amounts of free hormone available for (supposedly) passive diffusion and the levels required to efficiently occupy intracellular target receptors, the ‘free-hormone hypothesis’ raises doubts as a basis for a physiologically relevant (“true”) vitamin D status.

Total 25(OH)D is currently measured by LC-MS/MS (Institute of Standards and Technology and the Centers for Disease Control and Prevention). A variety of immunoassays are used to determine concentrations of total 25(OH)D and other vitamin D metabolites. However, all these methods produce highly variable results, likely due to the need for a releasing of the vitamin D metabolites from their carrier proteins. This comprises a risk of a loss of vitamin D through binding to vessel surfaces so that a falsely (low) vitamin D level is determined. Affinity chromatography studies using immobilized DBP, and solubilized rabbit kidney membranes have identified the co-receptors for the uptake of 25(OH)D₃-DBP complex in kidney tubules, namely a 600-kDa protein (megalin) and a 460-kDa protein (cubilin). These studies show a Ca²-dependent binding of DBP to cubilin. The authors do not suggest that the binding of DBP to either co-receptor is preceded, influenced or dependent on a formed complex of DBP and 25(OH)D₃, nor on the type of vitamin D metabolite bound by DBP (Nykjaer et al, Cubilin dysfunction causes abnormal metabolism of the steroid hormone 25(OH) vitamin D₃, Proc Natl Acad Sci USA. 2001 Nov. 20; 98(24): 13895-13900).

Megalin has three domains: a 4400 amino acid extracellular amino-terminal domain, a 22 amino acid transmembrane domain and a 213 amino acid carboxy-terminal cytoplasmic tail, indicating that it is a type I cell-surface receptor. The extracellular domain contains four cysteine rich clusters of LDLR type A (complement-type) repeats. The complement type repeat consists of approximately 40 amino acids containing six cysteine residues and the SDE (Ser-Asp-Glu) motif responsible for high-affinity binding of positively charged sequences in ligands for LDLR. The four cysteine-rich clusters are flanked by epidermal growth factor (EGF)-type repeats and spacer regions containing YWTD (Tyr-Trp-Thr-Asp) motifs which are responsible for pH-dependent dissociation of ligands in endosomal compartments. The cytoplasmic domain of megalin contains three tetra-amino-acid NPXY motifs, which are essential for endocytosis of the ligand-receptor complex via clathrin-coated pits.

Megalin has diverse types of ligands: vitamin-binding proteins and other binding proteins, apolipoproteins, hormones and hormone precursors, drugs and toxins, enzyme and enzyme inhibitors, immune-and stress-response-related proteins, and others including calcium. Megalin knockout mice are unable to recover DBP from the glomerular filtrate and lose it together with its vitamin D cargo in urine. As a consequence, megalin knockout mice are unable to adequately metabolize 25(OH)D to 1,25(OH)₂D resulting in a bone phenotype that resembles vitamin D-deficient rickets.

Cubilin has been identified as a receptor for intrinsic factor-B12 (IF-B) complex in the terminal ileum. It is a 460 kDa receptor with no transmembrane domain and no signals for endocytosis. Cubilin contains 27 CUB domains responsible for the ligand binding and eight EGF-type repeats preceded by a stretch of 110 amino acids, where the N-terminal region appears essential for membrane anchoring. A direct association between cubilin and megalin has been demonstrated, whereby molecular cooperation provides the basis for internalization of ligands bound to cubilin. Thus, cubilin binding ligands may undergo megalin-mediated endocytosis, unload its cargo in lysosomes and recycle back to the plasma membrane together with megalin.

Although megalin-dependent uptake of DBP has a clear role in renal vitamin D endocrinology, it is not yet clear whether a similar mechanism is present in other vitamin D target tissues. Outside the kidney, megalin is expressed by several tissues including the placenta, mammary gland, and parathyroid glands, which are known to have 1α-hydroxylase activity, suggesting an extra-renal DBP-megalin interaction.

The present disclosure describes a method of determining a vitamin D status based on the detection and quantitation of a complex comprising DBP, vitamin D and either one of megalin and cubilin or both. Different to the prior art, the present method requires no specific release of the vitamin D metabolites from DBP and only the fraction of vitamin D metabolites will be evaluated that is subject to endocytosis and activation. Without wishing to be bound by any theory, the measurement of DBP bound to megalin provides a parameter for endocytable vitamin D metabolites. The provided method can therefore be used to establish a vitamin D status which corresponds to the physiologically active and endocytable vitamin D concentration in serum or plasma.

With the present method, the various vitamin D metabolites, in particular 25(OH)D₃ can be discriminated as well as the changes in total serum concentrations of 25OHD, 1,25(OH)₂D and DBP in subjects following supplementation with either cholecalciferol (vitamin D₃) or ergocalciferol (vitamin D₂). Supplementation will no longer interfere with the measurement of active vitamin D molecules that will be subject to endocytosis and hydroxylation. The fraction of truly bioavailable vitamin D molecules can therefore easily be discerned from the supplemented vitamin D₂ or vitamin D molecules bound by other proteins in the circulation.

Immunonephelometry can be used to measure the DBP concentration in a sample. Immunonephelometry quantifies the scattering of an incident light source by large soluble antigen-antibody complexes under conditions of a moderate excess of antibody. Under these conditions, the complexes form a stable lattice, and a direct linear relationship is established between an increasing concentration of antigen and the increase in scattered light intensity. Automated nephelometers provide sensitive and precise measurements of DBP concentration in a rapid manner and with minimal requirements for technical skill. These can be used in clinical chemistry laboratories to analyze DBP concentrations. In a preferred embodiment, only the ternary complex formed by DBP, vitamin D and megalin is recognized by the antibody.

In another aspect, immunoturbidimetry can be used to measure the DBP concentration in a sample. Turbidimetry is the process of measuring the loss of intensity of transmitted light due to the scattering effect of particles suspended in it. Light is passed through a filter creating a light of known wavelength which is then passed through, for example, a cuvette containing a solution. A photoelectric cell collects the light which passes through the cuvette. A measurement is then given for the amount of absorbed light. Immunoturbidimetry is a variant in which an antigen-antibody reaction takes place. The antigen-antibody complexes are particles which can be optically detected by a photometer. In a preferred embodiment, the antibody recognizes a ternary complex formed by DBP, vitamin D and megalin. In another aspect, nanoparticles coated with megalin, or fragments thereof are contacted with a sample containing DBP/vitamin D complexes. Increasing concentrations of DBP/vitamin D/megalin ternary complex in the sample result in increased turbidity.

### EXAMPLES

### Example 1

Complex biological functions emerge through intricate protein-protein interaction networks. An important class of protein-protein interaction corresponds to peptide-mediated interactions, in which a short peptide stretch from one partner interacts with a large protein surface from the other partner. Protein-peptide interactions are typically of low affinity and involved in regulatory mechanisms, dynamically reshaping protein interaction networks. Due to the relatively small interaction surface, modulation of protein-peptide interactions has been considered feasible and highly attractive, for example, for therapeutic purposes. Unfortunately, the number of available 3D structures of protein-peptide interfaces is very limited. However, there was limited or no information regarding the interaction of DBP with other proteins, in particular, megalin/LRP2 (UniProtKB/Swiss-Prot, protein accession number P98164, human) in the context of vitamin D transport and metabolism. In order to examine the potential interaction of DBP with megalin at the peptide level, the PepSite2 program (http://pepsite2.russelllab.org) was used to predict peptide-binding spots. The PepSite method relies on preferred peptide-binding environments calculated from a set of known protein-peptide 3D structures, combined with distance constraints derived from known peptides. According to the PepSite prediction, megalin likely interacts with human vitamin D-binding protein through ligand-binding repeats 9, 13-15, 16, 19, 22-25; 27 and 29 located at domains 2 and 3 (see FIG. 1A)

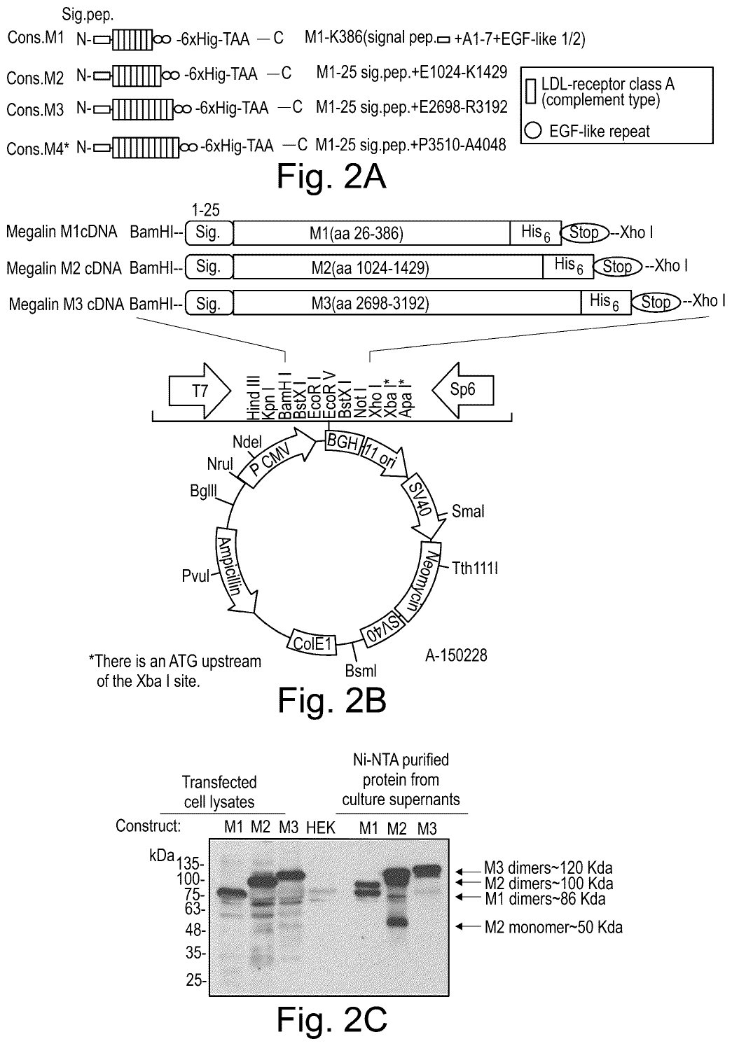

In agreement with the above prediction, the present inventors selected cysteine-rich complement-type ligand binding repeats (LDLR class A) for analysis of DBP-megalin interaction. EGF-like modules were also included as they are considered important for receptor folding and dissociation of ligands in endosomal compartment. A signal peptide (M1-G25) was introduced at the N-termini of complement-type repeats to allow sorting to the secretory pathway. A C-terminal 6xHis-tag was added for affinity purification and protein analysis by co-immunoprecipitation and western blotting. The respective cDNA sequences were obtained by PCR and inserted into the mammalian expression vector pcDNA3.1. Since calcium is required for a correct folding of the LDLR-A domain, HEK cells were stable transfected with megalin cDNA sequences (M1, M2, M3) as they have endogenous calcium channels. Protein purification was done using the His-tag and a commercially available Ni-NTA resin. The cloning strategy for megalin fragments (M1, M2, M3) into the mammalian expression vector pcDNA3.1 is shown in FIGS. 2A-B.

Megalin M1 cDNA encoded the sequence of amino acids 26 to 386 of human LRP2/megalin (SEQ ID NO:08). The construct included cDNA encoding for an N-terminal 25 amino acid signal peptide and a C-terminal 6x histidine tag. The predicted molecular weight was about 43 kDa for the monomer and about 86 kDa for the dimer.

The HEK-expressed and secreted recombinant megalin M1 fragment had the following amino acid sequence (without signal sequences):

SEQ ID NO: 08
IPADWRCDGT KDCSDDADEI GCAVVTCQQG YFKCQSEGQC
IPNSWVCDQD QDCDDGSDER QDCSQSTCSS HQITCSNGQC
IPSEYRCDHV RDCPDGADEN DCQYPTCEQL TCDNGACYNT
SQKCDWKVDC RDSSDEINCT EICLHNEFSC GNGECIPRAY
VCDHDNDCQD GSDEHACNYP TCGGYQFTCP SGRCIYQNWV
CDGEDDCKDN GDEDGCESGP HDVHKCSPRE WSCPESGRCI
SIYKVCDGIL DCPGREDENN TSTGKYCSMT LCSALNCQYQ
CHETPYGGAC FCPPGYIINH NDSRTCVEFD DCQIWGICDQ
KCESRPGRHL CHCEEGYILE RGQYCK

Megalin M2 cDNA encoded the sequence of amino acids 1024-1429 of human LRP2/megalin (SEQ ID NO:09). The construct included cDNA encoding for an N-terminal 25 amino acid signal peptide and a C-terminal histidine(6x) tag. The predicted molecular weight was about 50 kDa for the monomer and about 100 kDa for the dimer. The HEK-expressed and secreted recombinant protein megalin M2 fragment had the following amino acid sequence (without signal sequences):

SEQ ID NO: 09
EQCGLFS FPCKNGRCVP NYYLCDGVDD CHDNSDEQLC
GTLNNTCSSS AFTCGHGECI PAHWRCDKRN DCVDGSDEHN
CPTHAPASCL DTQYTCDNHQ CISKNWVCDT DNDCGDGSDE
KNCNSTETCQ PSQFNCPNHR CIDLSFVCDG DKDCVDGSDE
VGCVLNCTAS QFKCASGDKC IGVTNRCDGV FDCSDNSDEA
GCPTRPPGMC HSDEFQCQED GICIPNFWEC DGHPDCLYGS
DEHNACVPKT CPSSYFHCDN GNCIHRAWLC DRDNDCGDMS
DEKDCPTQPF RCPSWQWQCL GHNICVNLSV VCDGIFDCPN
GTDESPLCNG NSCSDFNGGC THECVQEPFG AKCLCPLGFL
LANDSKTCED IDECDILGSC SQHCYNMRGS FRCSCDTGYM
LESDGRTCK

Megalin M3 cDNA encoded the sequence of amino acids 2698 to 3192 of human LRP2/megalin (SEQ ID NO: 10). The cDNA encoded an 25 amino acid signal peptide and a C-terminal 6x histidine tag. The predicted molecular weight was about 60 kDa for the monomer and 120 kDa for the dimer. The construct was expressed in HEK 293 mammalian cells. The secreted megalin M3 fragment. SEQ ID NO. 10 corresponds to following amino acid sequence (without signal sequences):

SEQ ID NO: 10
ERC GASSFTCSNG RCISEEWKCD NDNDCGDGSD EMESVCALHT
CSPTAFTCAN GRCVQYSYRC DYYNDCGDGS DEAGCLFRDC
NATTEFMCNN RRCIPREFIC NGVDNCHDNN TSDEKNCPDR
TCQSGYTKCH NSNICIPRVY LCDGDNDCGD NSDENPTYCT
THTCSSSEFQ CASGRCIPQH WYCDQETDCF DASDEPASCG
HSERTCLADE FKCDGGRCIP SEWICDGDND CGDMSDEDKR
HQCQNQNCSD SEFLCVNDRP PDRRCIPQSW VCDGDVDCTD
GYDENQNCTR RTCSENEFTC GYGLCIPKIF RCDRHNDCGD
YSDERGCLYQ TCQQNQFTCQ NGRCISKTFV CDEDNDCGDG
SDELMHLCHT PEPTCPPHEF KCDNGRCIEM MKLCNHLDDC
LDNSDEKGCG INECHDPSIS GCDHNCTDTL TSFYCSCRPG
YKLMSDKRTC VDIDECTEMP FVCSQKCENV IGSYICKCAP
GYLREPDGKT CR

Protein analysis of megalin M1, M2, M3 fragments was done by Western blotting employing an antibody against 6x His-tag. Cells transfected with above constructs (M1, M2, M3) were lysed and the lysate analyzed for the proteins with a His-tag. Megalin fragments were also purified from the cell supernatants using Ni/NTA resin, which bind proteins with a His-tag (right). The results are shown in FIG. 2C.

The Western blots show that megalin M1, M2, M3 fragments were expressed and secreted by HEK cells. The megalin fragments could be kept in solution. No precipitation was observed. The megalin fragments showed a tendency to form dimers but the molecular weights were all consistent with the predicted sizes. 2× Laemmli and 2× Urea sample buffers were used for analysis of megalin monomer and dimer formation, but no difference was observed by the use of these buffers. The M2 fragment was the one which could be mostly easily dissociated to the monomer.

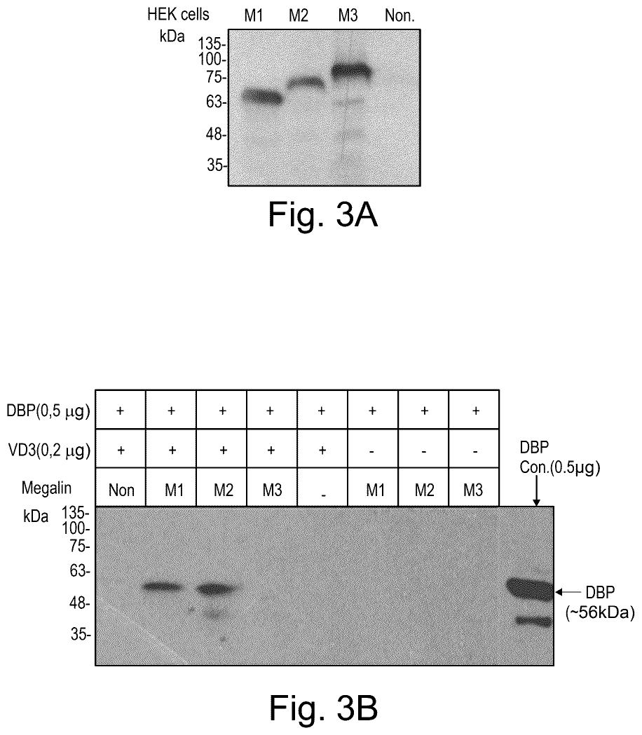

The megalin fragments were further examined for their interaction with DBP in the presence (+) or absence (−) of vitamin D₃ (VD₃). Co-immunoprecipitations were performed and the results are shown in FIGS. 3A,B. In brief, HEK cells were transfected with above cDNA constructs (M1, M2, M3) and cell lysates used for co-immunoprecipation. FIG. 3A shows a Western blot of cell lysates (20 μg) of HEK cells expressing M1, M2 and M3. Detection with an antibody against the His-tag. FIG. 3B shows the results of a co-immunoprecipitation with DBP (0.5 μg and VD₃ (0.2 μg).

The serum samples included DBP alone or in the presence of added vitamin D₃. Each sample was contacted with cell lysate containing megalin fragment. After incubation, the samples were contacted with beads coated with an antibody against His-tag and the DBP pulled down by centrifugation. The bound ligands (DBP) were dissociated from the beads and analyzed by western blot for the expression of DBP using an anti-DBP-antibody. As control, pure DBP alone was analyzed. FIG. 3B confirms that DBP could be pulled down with megalin M1 and M2 fragments. Notably, DBP could only be pulled down in the presence (+) of vitamin D₃. This indicates that the megalin fragments M1 and M2 do not interact with DBP alone but only when the DBP is occupied with a vitamin D metabolite. In other words, a ternary complex comprising vitamin D binding protein, vitamin D metabolite and megalin fragment was formed in vitro, and no interaction took place between megalin M1 or M2 fragments and DBP alone.

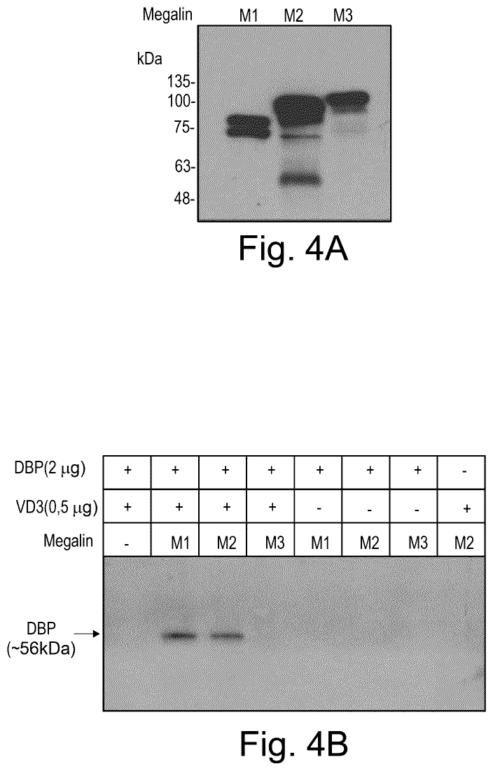

Additional co-immunoprecipitations show the in vitro interaction between DBP and Ni-NTA agarose purified soluble megalin M1, M2, M3 fragments (3 μg) in a similar set-up as above. The results are shown in FIGS. 4A,B. FIG. 4A is a western blot of purified megalin M1, M2, M3 fragments from culture supernatants (3 μg), secreted by HEK cells transfected with cDNA for megalin M1, M2 and M3 fragments; detection by anti-His-antibodies. FIG. 4B shows a western blot with the results of a co-immunoprecipitation with purified megalin M1, M2, M3 fragments. The serum samples contained DBP alone or in the presence (+) of vitamin D₃. After incubation, the samples were contacted with beads coated with an antibody against 6xHis-tag and pulled down by centrifugation. Bound proteins were dissociated from the beads and analyzed by Western blotting using an antibody against vitamin D binding protein. Again, FIG. 4B confirms that DBP could be pull down with purified soluble M1 and M2 fragments. DBP was pulled down only in the presence (+) of vitamin D₃. This indicates that soluble purified megalin fragments M1 and M2 do not interact with DBP unless occupied with vitamin D₃. Ternary complexes comprising DBP, vitamin D₃ and purified megalin M1 or M2 fragments were formed in vitro. No interaction or binding was observed between soluble purified megalin M1 or M2 fragments and DBP alone.

### Example 2

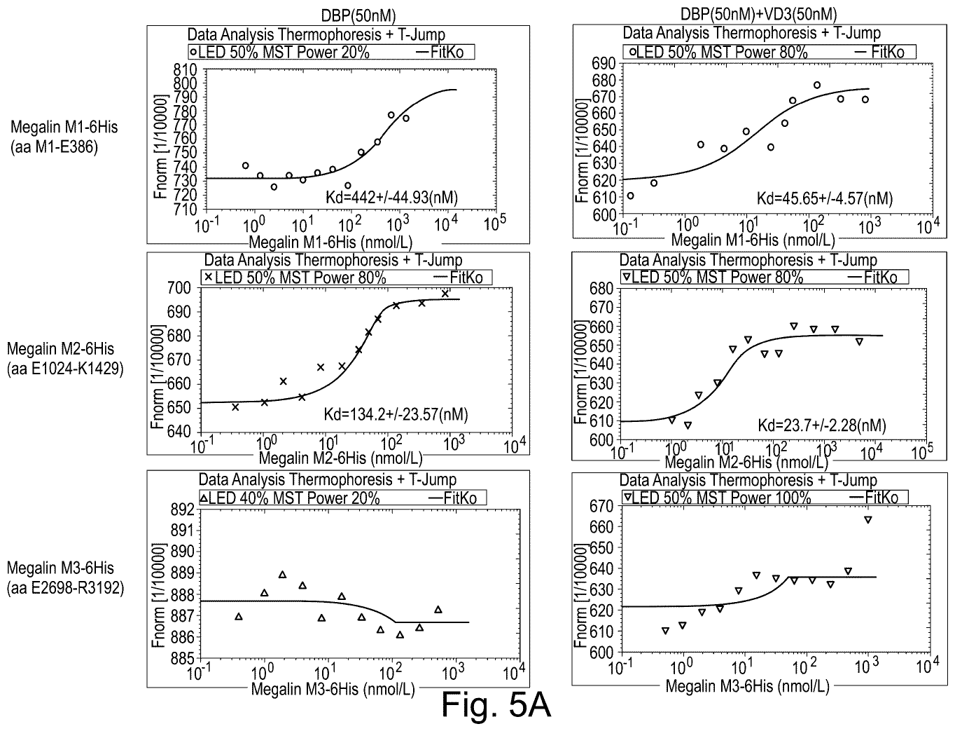

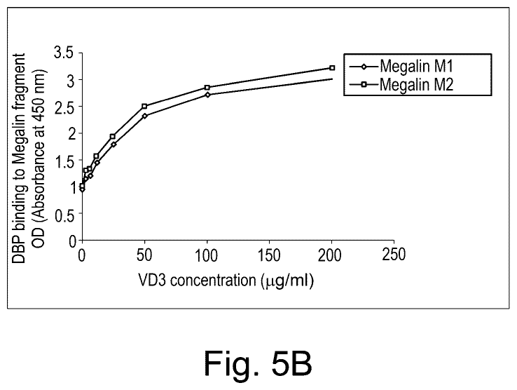

For further characterization of the formed complex of purified soluble megalin M1 or M2 fragments and DBP alone or occupied by vitamin D metabolite, the obtained complexes were further analyzed by microscale thermophoresis and results are shown in FIG. 5A. Microscale thermophoresis (MST) examines the directed movement of particles in a microscopic temperature gradient (thermophoresis). Any change of the hydration shell of biomolecules due to changes in their conformation results in a relative change of the movement along the temperature gradient. This principle can be used to determine the binding affinity of two molecules. This technique allows in particular an examination of interactions in solution without any immobilization on a surface. A spatial temperature difference leads to a depletion of molecule concentration in the region of elevated temperature, which can be then determined. Thermophoresis is usually performed with fluorescently labeled molecules.

The difference in the molecule's thermophoresis can further be to quantify the binding strength under constant buffer conditions. The thermophoretic movement of the fluorescently labeled molecule is measured by monitoring the fluorescence distribution inside a capillary. The microscopic temperature gradient is generated by an IR-Laser, which is focused into the capillary and absorbed in water. The temperature of the aqueous solution in the laser spot region therefore increases. A homogeneous fluorescence distribution is observed inside the capillary prior the IR-Laser is switched on. When the IR-Laser is switched on, a new fluorescence distribution is established. The thermal relaxation time is fast and induces a binding-dependent drop in the fluorescence of the dye due to its local environmental-dependent response to the temperature step increase. Molecules move then from the locally heated region to the outer cold regions. The local concentration of molecules decreases in the heated region until it reaches a steady-state distribution.

The normalized fluorescence (Fnorm) measures a concentration ratio, with consideration of the temperature step increase. Due to the linearity of the fluorescence intensity and the thermophoretic depletion, the normalized fluorescence from the unbound molecule and the bound complex superpose linearly. Quantitative binding parameters were obtained using serial dilutions of the binding substrate. By plotting Fnorm against the logarithm of the different concentrations of the dilution series, a sigmoidal binding curve is obtained. This binding curve can directly be fitted with the non-linear solution of the law of mass action, with the dissociation constant Kd as result.

In brief, purified DBP was labeled with the red fluorescent dye NT-647 using Monolith Protein Labeling Kit Red (NanoTemper Technologies, Munich, Germany). Ni-NTA-purified soluble megalin fragments M1, M2, and M3 fragments were titrated in the range from 0.488 to 1000 nmol/L. A Monolith NT.115 device (NanoTemper Technologies) and the NT Analysis software version 1.427 (NanoTemper Technologies) were used for measurements.

Microscale thermophoretic analysis of the binding between His-Tag purified soluble megalin M1, M2, and M3 fragments and DBP in the absence or in the presence of vitamin D₃ were performed. The results are shown in FIG. 5A. In line with the above co-immunoprecipitation experiments, M1 and M2 fragments interacted with DBP but M3 did not. The dissociation constants were calculated for each experiment. Both megalin M1 and M2 fragments showed weak binding to DBP in the absence of 25-OH vitamin D₃ (M1/DBP: Kd=442+/−44.93 nM; M2/DBP: 134.2+/−23.57 nM)). In contrast, high binding affinity was found for megalin fragments in the presence of 25(OH)D₃ as the dissociation constant Kd was markedly lower (M1/DBP/VD₃: Kd=45.65+/−4.57 nM; M2/DBP/VD₃: 23.7+/−2.28 nM). We also observed increased binding of DBP to purified soluble megalin fragments M1 or M2 in the presence of 25(OH)D.

The results are good evidence of a 25(OH)D₃-dependent binding DBP to megalin. Megalin M2 fragment had a higher affinity for the complex of DBP: 25(OH)D₃ and was used therefore in further studies.

The impact of the 25(OH)D₃ concentration on the binding of soluble megalin M1 and M2 fragment to DBP was further assessed using an ELISA which already represents an embodiment of the proposed new complex binding assay for the effective vitamin D status. The results are shown in FIG. 5B. In brief, a microtiter plate was coated with purified soluble megalin M1 or M2 fragment (1 μg/100 μl PBS). Mixtures of DBP and 25(OH)D₃ were prepared using 1 μg DBP and serial dilutions of 25(OH)D₃ (VD₃) in PBS (0, 0.3125, 0.625, 1.25, 2.5, 5, 10, 20 μg). Each mixture was applied to the coated wells, incubated at physiological conditions to allow formation of a ternary complex, and washed. Detection and quantification of DBP bound by megalin (soluble fragments M1 and M2) was carried out by using a polyclonal rabbit antibody against DBP and HRP-conjugated donkey anti-rabbit antibody. Absorbance was read at 450 nm.

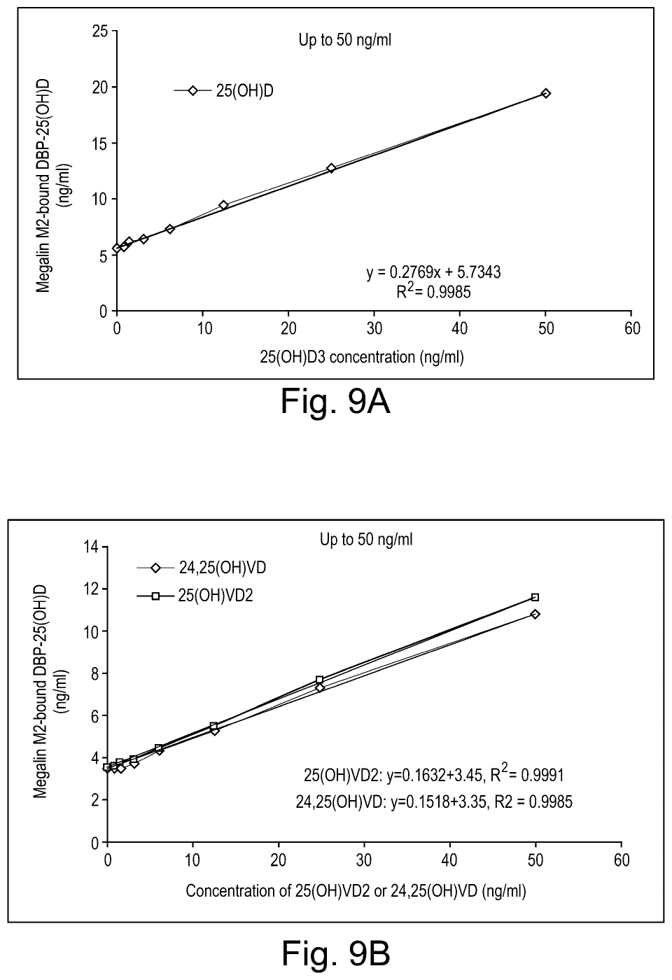

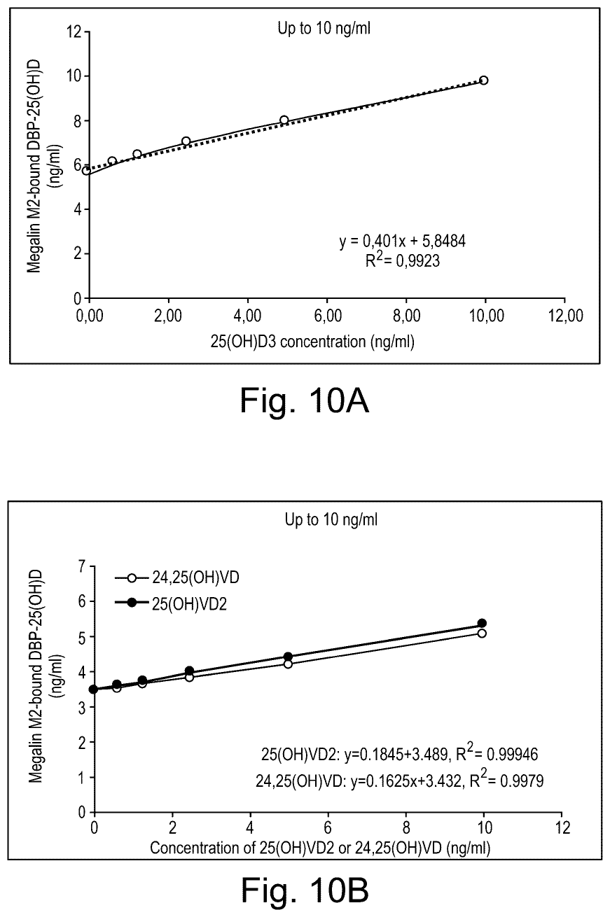

As shown in FIG. 5B, the binding of soluble megalin M1 and M2 fragments to DBP was dependent on the 25-hydroxy-vitamin D₃. More precisely, the absorbance is linear proportional to the vitamin D₃ concentration as shown in FIG. 9A and FIG. 10A and this relation is not impacted by increasing concentrations of 25(OH)VDBP or 24,25(OH)VD (cf. FIG. 9B, 10B). Importantly, these experiments demonstrate a proof of principle for easy and reliable vitamin D measurements based on the ternary complex.

### Example 3

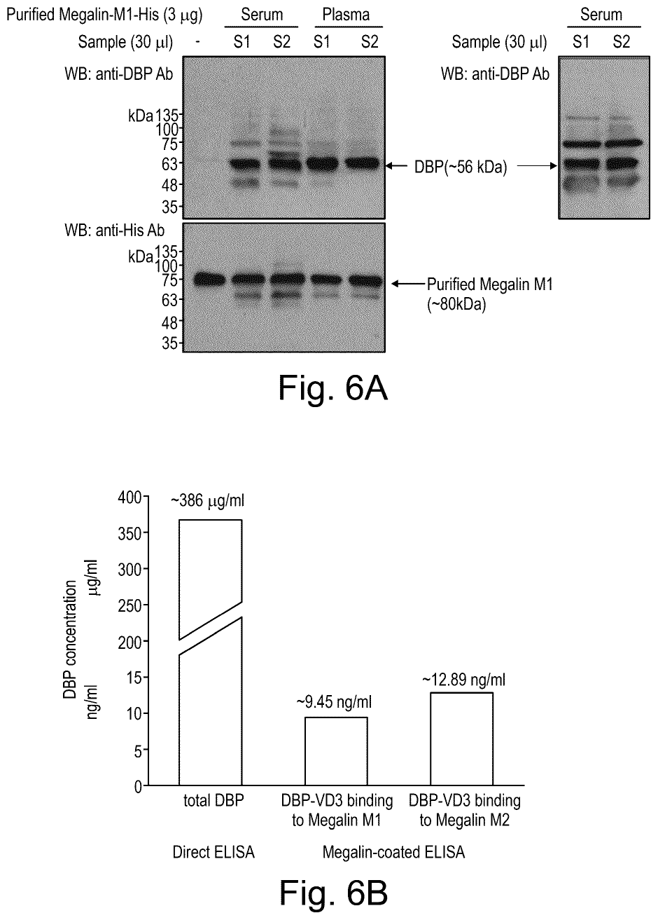

The binding properties of the various megalin soluble fragments were further analyzed by co-immunoprecipitation using samples of serum and plasma from human subjects. The results are shown in FIG. 6A. Purified soluble megalin M1 protein was mixed with two human serum or plasma samples to allow interaction between soluble megalin M1 fragment and endogenous DBP present in the sample. Samples were incubated with Ni-NTA resin to allow interaction of megalin M1/DBP to the resin. After washing the resin, bound complex was eluted from the resin and assayed by western blotting. FIG. 6A (right) shows that DBP was present in both samples and could be detected using a specific antibody. FIG. 6A (left, upper blot) shows that purified soluble megalin M1 fragment interacted in solution with DBP in all samples, as demonstrated by the presence of pulled down DBP. Of note, using a control sample containing serum only but no megalin protein, no DBP was detectable. The presence of purified megalin M1 protein was detected in all samples using an anti-6xHis-tag antibody (FIG. 6A, left, lower blot).

In summary, the co-immunoprecipitations are proof that the complex of DBP and 25(OH)D₃ can specifically be bound and isolated from plasma or serum using a suitable soluble megalin fragment. The megalin portion with amino acids 26-386 and 1024-1429 have been shown to be involved in the formation of a ternary complex with DBP and 25(OH)D₃. The external megalin region with amino acids 2698-R3192 did not participate in the binding under the described conditions.

The present application comprises representative amino acid sequences of megalin which can be used in the binding of DBP. Those can further be used for determining the status of endocytic or activatable vitamin D in bodily fluids. The status of endocytable vitamin D overrules any status for “free vitamin D” or “total vitamin D” as there will be no need for distinguishing between “free” or “total” from the “physiological status of vitamin D available for endocytosis and 1α-hydroxylation”. Conventional methods usually do not determine the physiologically relevant vitamin D status since they cannot analyze the metabolites ready for processing to the active hormone. Thus, the conventional methods are insufficient whereas the present method is directed to the status of circulating prohormone which can and will be hydroxylated in the kidney, and probably in other tissues, giving rise to the active hormone.

### Example 4

For detailed characterization of the proportion of the ternary complex of DBP, vitamin D₃ and megalin (M1 and M2 fragments) an ELISA was developed using megalin-coated plates. The total amount of DBP in the serum sample was determined using a commercial ELISA for DBP. The results are shown in FIG. 6B. The assay shows that a minor proportion (0.0028%) only of the total amount of DBP in serum has bound hydroxylated vitamin D₃ and can be bound by megalin. Using megalin M1 fragment, the assay yielded a concentration value of 9.45 ng complex/mL serum. In the case of megalin M2 fragment, a value of about 12.89 ng complex/mL serum was obtained. This is evidence that the megalin fragment did only DBP when in a complex with the prohormone. Megalin can be thus regarded a means for detection and quantification of the physiologically activated prohormone (bound to DBP) in a clinical sample.

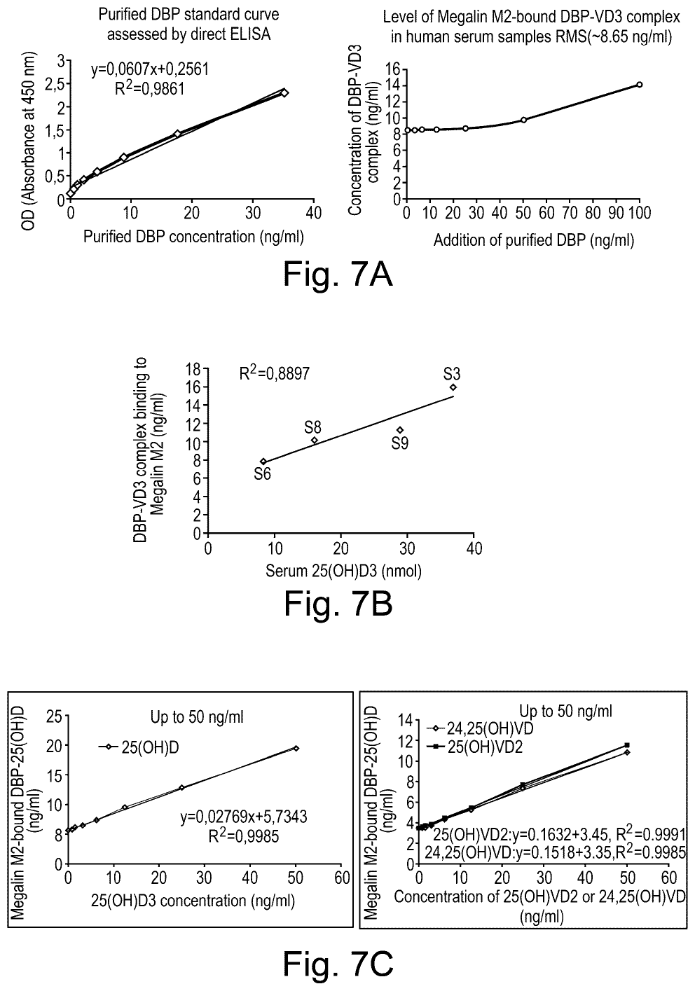

We noted that it was sometimes necessary to add an amount of purified DBP (DBP, from about 5 to 25 ng/ml) to induce a ternary complex between megalin, DBP and vitamin D metabolite in human serum. In order to assess the effect of added DBP on the formation of ternary complex we also performed ELISAs with increasing amounts of purified vitamin D binding protein. The results are shown in FIG. 7A. In the serum sample (RMS), addition of purified DBP in a dose range up to 25 ng/ml did not affect the binding of the complex DBP/VD₃ to purified soluble megalin M2 fragment. The initial value of 8.65 ng/ml remained unchanged independently of added purified DBP. A slight increase in complex-megalin interaction was observed when adding high amounts of purified DBP up to 40 ng/ml. Above this limit, the ternary complex was formed exponentially. Within limits, the measured levels of ternary complex of DBP-VD₃-megalin do therefore not depend on the amount of added purified vitamin D binding protein.

For comparison, five serum samples (S3, S6, S8, S9, RMS) from different subjects were analyzed to determine the concentration of total DBP (DBP), 25(OH)D₃ and ternary complex of DBP:25(OH)VD:M2. DBP was determined by ELISA. The 25(OH)D₃ content was quantified conventionally by an independent laboratory. The ternary complex DBP:25(OH)VD:M2 megalin was analyzed by a DBP:VD3:megalin ELISA according to the disclosure. Table 1 below summarizes the results for comparison.

TABLE 1

| Serum | Total DBP | 25(OH)VD₃ | | DBP:25(OH)VD:M2 |
|---|---|---|---|---|
| sample | (μg/mL) | (nmol/L)/(ng/mL) | | (ng/mL) |
| S3 | 576.7 ± 69.8 | 92 | 36.86 | 15.9 ± 2.4 |
| S6 | 208.1 ± 36.7 | 21 | 8.4 | 7.8 ± 1.1 |
| S8 | 352.2 ± 65.3 | 40 | 16 | 10.2 ± 1.4 |
| S9 | 732.5 ± 83.9 | 72 | 28.84 | 11.3 ± 1.8 |
| RMS | 424.9 ± 53.9 | — | — | 8.6 ± 1.5 |

Current standards for healthy control (interquartile) range values are for i) serum DBP 193.5-4345.0 μg/ml (median 423.5 μg/ml; 354.1-586 μg/ml), and for ii) serum 25(OH)D₃ 30-100 ng/mL (normal range), 20-30 ng/ml (insufficiency), <20 ng/ml (deficiency). Accordingly, sample S3 was considered to have normal levels of 25(OH)D₃; S9, insufficient; S8 and S6, deficient levels. In other words, from higher to lower levels of 25(OH)D₃: S3>S9>S8>S6. From the results it could be concluded that the level of total DBP seemed not to correlate with the level of 25(OH)D₃ level in human serum samples.

Although a direct comparison is strictly not possible since different analytes were measured by different methods, it is noteworthy that a correlation in the levels of ternary complex DBP:25(OH)VD:M2 megalin could be found in the samples when compared to 25(OH)D₃ values; from higher to lower levels of ternary complex S3>S9>S8>S6, identical as determined for 25(OH)D₃ levels. This further supports the notion that megalin only interacts in vitro with DBP when the prohormone is bound, providing a new status of physiologically active, endocytable vitamin D, which describes the bioavailability of the prohormone in the circulation.

The correlation between 25(OH)VD₃ serum levels and ternary complex was furhter analyzed; see graph in FIG. 7B. A linear correlation between serum levels of 25(OH)VD₃ and DBP:25(OH)VD:M2 megalin was determined (R²=0.8897) indicating that the method can provide accurate information that can be compared or related to standard values so as to obtain a measurement of the activatable vitamin D status of a subject.

Moreover, the linearity range of the interaction between megalin and DBP was examined using the claimed method for 25-hydroxyvitamin D₃ (25(OH)VD₃), 25-hydroxyvitamin D₂ (25(OH)VDBP) and 24,25-dihydroxyvitamin D (24,25(OH)VD). ELISAs for the complex of DBP:VD:megalin were performed as disclosed. In brief: a microtitre plate coated with purified megalin M2 fragment was incubated with a mixture of DBP and serial dilutions of 25(OH)VD₂, 25(OH)VD₃ or 24,25(OH)VD. For determination of the ternary complex, a polyclonal rabbit anti-DBP antibody and a HRP-conjugated donkey anti-rabbit antibody were used. The results are shown in FIG. 7C. For all vitamin D metabolites, the assay was linear up to 50 ng/ml. The determined sensitivity limit was 2.0 ng/ml.

The binding properties of DBP and 25(OH)VD₃, 25(OH)VD₂, 24,25(OH)VD and 3C-epimer of 25(OH)D₃ on the one hand and megalin on the other was further examined using microscale thermophoresis. Results are shown in FIGS. 8A,B. The purified DBP was labeled with red fluorescent dye NT-647. The vitamin D metabolites were titrated with different concentrations. The dissociation constant Kd was calculated for every interaction partner. The binding affinity was assessed via the obtained dissociation constant Kd. The results show that the interaction affinity of DBP to 25(OH)VD₃ (Kd=1.88+/−0.28 nM) or 25(OH)VDBP (Kd=2.81+/−0.681 nM) were comparable, whereas the interaction to 24,25(OH)VD appeared to be slightly weaker. The 3C-epi25(OH)VD₃ bound most strongly.

The affinity of DBP when interacting with 25(OH)VD₃, 25(OH)VD₂, or 24,25-24,25(OH)VD, 3C-epimer of 25(OH)D₃ and megalin M2 was also analyzed by microscale thermophoresis. The results are shown in FIG. 8B. Purified DBP (50 nM) was labeled with the red fluorescent dye NT-647 and mixed with either vitamin D metabolite (37.8 nM). Purified megalin M2 was titrated in different concentrations. The dissociation constant Kd was calculated for every condition. The binding affinity was assessed by the dissociation constant Kd. The analyses show that the binding affinity of megalin M2 fragment to the complex DBP:25(OH)VD₃ is much stronger (Kd=33.7+/−20.7 nM) than to the complex DBP:25(OH)VD₂ (Kd=160+/−22.7 nM), or −/DBP-24,25VD (Kd=171+/−24.6 nM). A very high affinity was observed for the 3C epimer which will require further investigation.

These experiments demonstrate that the binding of megalin to DBP takes only place when bound to the prohormone. This ternary complex can be detected and quantified by any method for determination of proteins. In view of the above, the use of megalin allows for a reliable measurement of the physiologically activated vitamin D, say 25-hydroxyvitamin D₃, as this represents the most active form of vitamin D in the circulation. The disclosed method provides for differential measurement of other vitamin D metabolites which is also relevant in view of the vitamin DBP in food supplements.

Megalin or fragments thereof are used to discern the effective vitamin D status. The described megalin fragments can easily be produced by recombinant methods and even by chemically synthesized. The described megalin fragments remained soluble in aqueous solution so that the conditions close to physiological can be used. There is no longer a need for a release of vitamin D from its binding partners, as required by the prior art methods. Thus, no solvents or surfactants for displacement of the vitamin D from its binding partners are needed. Accordingly, the measurement of the vitamin D status will not be interfered by non-physiological chemicals. No purification steps or time consuming and costly techniques such as LC-MS are necessary. A direct and fast determination of the effective vitamin D status can be done immediately after sample collection. The disclosed principles can further be easily adapted to available platforms and automats.

As other ligands than DBP can also bind to megalin, we have further mapped more closely the binding regions or epitope within the M2 region. The results are contained in the provided sequence for the binding epitopes. Minimum binding epitopes have further been tested.

### Example 5

Production of recombinant megalin fragments. Megalin (LRP2) cDNA fragments were amplified by RT-PCR using mRNA from Caco-2 cells (human colon carcinoma epithelial cells). For cDNA transcription synthesis Maxima H Minus First strand cDNA synthesis kit (ThermoScientific) was used. This kit allows synthesis of cDNA up to 20 kb. Oligo dt18 primer and 65C were used. For PCR reactions Platinum PCR Supermix high fidelity PCR kit (Invitrogen) was used. Megalin M1 cDNA: 1158 bp; Megalin M2 CDNA: 1215 bp; Megalin M3 cDNA: 1482 bp.

Megalin cDNA fragments were cloned with a C-terminal His-Tag into pcDNA3.1, and the cloned plasmids transfected into mammalian HEK293 cells. The following cloning strategy was applied to clone megalin M2 cDNA encoding for a sequence of amino acids 1024-1429 of human LRP2/megalin (SEQ ID NO:09) and resulted in the construct having: signal peptide (25 amino acids)+E1024-K1429+6x His tag. The cloning was done using PCR-generated sequences.

Stable cell line selection was carried out using neomycin (G418, 800 μg/ml). Cell culture supernatants or cell lysates were purified by Ni-NTA resin. Analysis of megalin protein M1, M2, M3 fragments having a 6xHis-Tag was performed by Western blot with an antibody against 6xHis-tag (Cohesion Biosciences). Cell culture supernatants or cell lysates were purified with Ni-NTA agarose (ThermoFischer Scientific). Cell lysates or culture supernatant (0.5-2 mL) were incubated with 50 μl of pre-equilibrated Ni-NTA resin at 4° C. overnight. The resin was washed 3 times with H-buffer+20 mM imidazole. The megalin protein complex was eluted with H-buffer containing 200 mM imidazole and buffer exchanged with PBS 1X. Protein solutions were lyophilized or kept at 4° C. prior to use.

The fragment M2 was further mapped for major binding epitopes of Megalin ligands within the M2 region, the region of megalin which binds human DBP. The binding sites of other megalin ligands were removed, and the epitopes for binding of human DBP identified. It is important that the minimum epitopes do not overlap with the binding epitopes of other ligands off megalin. The results are shown in the Tables below.

TABLE 2
Major binding epitopes of Megalin ligands within M2 region (aa E1024-K1429)

| | |
|---|---|
| Human DBP | Epitope 1 G-C-T-H-E-C-V-Q-E-P-F-G-A-K-C |
| | Epitope 2 D-N-G-N-C-I-H-R-A-W-L-C-D-R-D |
| Rabbit-anti-hDBP-IgG Ab | A19-A20 Y-T-C-D-N-H-Q-C-I-S-K-N-W-V-C-D-T-D-N-D |
| (Immundiagnostik) | |
| Human Ig κC | C8: M-S-D-E-K-D-C-P-T-Q-P-F-R-C-P |
| Human C1q | A19 Y-T-C-D-N-H-Q-C-I-S-K-N-W-V-C |
| | B22 E-C-D-G-H-P-D-C-L-Y-G-S-D-E-H |
| Human Albumin-His-tag | Epitope 1 (622): E-C-D-G-H-P-D-C-L-Y-G-S-D-E-H |
| | Epitope 2 (C24-D1): F-L-L-A-N-D-S-K-T-C-E-D-I-D-E-C-D-I-L-G |
| Human RAP | Epitope 1 (B18-619): M-C-H-S-D-E-F-Q-C-Q-E-D-G-I-C-I-P-N-F-W |
| | Epitope 2 (D8): C-D-T-G-Y-M-L-E-S-D-G-R-T-C-K |
| Human ApoE | Epitope 1 (AH): C-G-H-G-E-C-I-P-A-H-W-R-C-D-K |
| | Epitope 2 (D₃): C-D-I-L-G-S-C-S-Q-H-C-Y-N-M-R |
| | Epitope 2 (D8): C-D-T-G-Y-M-L-E-S-D-G-R-T-C-K |
| Human PTH | Epitope 1 (B22): E-C-D-G-H-P-D-C-L-Y-G-S-D-E-H |

TABLE 3
Epitope mapping of ligands binding to the human Megalin M2 region (aa E1024-K1429)

| | |
|---|---|
| LDL-receptor class A8 | EQCGLFS FPCKNGRCVPNYYLCDGVDDCHDNSDEQLCG |
| (1024-1061) | |
| LDL-receptor class A9 (1066-1102) | 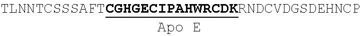 |
| LDL-receptor class A10 (1108-1144) | 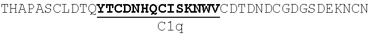 |
| LDL-receptor class A11 | STETCQPSQFNCPNHRCIDLSFVCDG DKDCVDGSDEVGCV |
| (1148-1184) | |
| LDL-receptor class A12 | LNCTASQFKCASGDKCIGVTNRCDGVFDCSDNSDEAGCP |
| (1186-1223) | |
| LDL-receptor class A13 (1229-1267) | 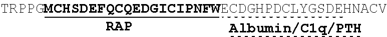 |
| LDL-receptor class A14 | PJTCPSSYFHCDNGNCIHRAWLCDRDNDCGDMSDEKDCP |
| (1270-1306) | DPB lg κC |
| LDL-receptor class A15 | TQPFRCPSWQWQCLGHNICVNLSWCDGIFDCPNGTDESPLCN |
| (1304-1349) | lg κC |
| Special domain (1350-1389) | 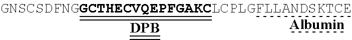 |
| EGF-like1; calcium-binding (1390-1429) | 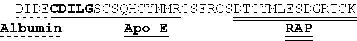 |

The minimum epitopes within the megalin M2 region for binding of human DBP have been listed in Table 3 below.

TABLE 4
List of Megalin-M2-peptides cloned and expressed as fusion
polypeptides with furin

| Name | Peptide Sequence | Peptide cDNA |
|---|---|---|
| Peptide 1-15 aa | DNGNCIHRAWLCDRD | GACAACGGAAACTGCATCCAC |
| | | AGGGCATGGCTCTGTGATCGG |
| | | GAC |
| Peptide 2-15 aa | GCTHECVQEPFGAKC | GGTTGTACTCACGAGTGTGTT |
| | | CAAGAGCCCTTTGGGGCTAAA |
| | | TGC |
| Peptide 3-30 aa | KTCPSSYFHCDNGNCIHR | aagacttgcccttcatcatatttccactgtgac |
| | AWLCDRDNDCGD | aacggaaactgcatccacagggcatggctc |
| | | tgtgatcgggacaatgactgcggggat |
| Peptide 4-30 aa | SDFNGGCTHECVQEPFGA | tcagatttcaatggtggttgtactcacgagtgt |
| | KCLCPLGFLLAN | gttcaagagccctttggggctaaatgcctatg |
| | | tccattgggattcttacttgccaat |

### Example 6

Megalin complex ELISA binding assay. Microtiter plates were coated with soluble purified megalin M1 or M2 fragment (1 μg/100 μl diluted in PBS1X) by incubation at RT for 2 h. Serial dilutions of 25-hydroxvitamin D₃ (VD₃) 0, 0.3125, 0.625, 1.25, 2.5, 5, 10, 20 ng in 100 μl PBS were mixed each with 1 μg DBP (DBP). The mixture was incubated at 37° C. for 1 h. Unspecific binding sites were blocked with blocking buffer (1% BSA in PBS1X) at RT for 1 h. The DBP-VD₃ mixture or serum samples were added to the megalin-coated wells and incubated at 4° C. overnight. After washing with PBST-buffer (0.05% Tween 20 in PBS) 100 μl rabbit-anti-DBP antibody was added (1:3500 diluted in blocking buffer) and incubated at R.T for 2 h. After washing with PBST, 100 μl of HRP-conjugated donkey anti-rabbit 2nd antibody (1:500 diluted in blocking buffer) was added and incubated at 37° C. for 1 h. 100 μl substrate reagent A+B (1:1) (R&D) was added and incubated at R.T for 30 min. Then, 100 μl stop solution was added to the wells. Absorbance was read at 450 nm. The values were compared to standard values of known 25-hydroxvitamin D₃ concentration.

### Example 7

Co-immunoprecipitation of DBP from human serum with recombinant purified soluble megalin. Ni-NTA purified soluble megalin M1 or M2 protein fragment (3 μg) was first mixed with human serum or plasma samples (30 μl). These samples were incubated with 50 μl of pre-equilibrated Ni-NTA resin at 4° C. overnight. The resin was washed 3 times with H-buffer+20 mM imidazole. The bound complex was eluted with H-buffer containing 200 mM imidazole and analyzed by Western blot with a polyclonal rabbit antibody against DBP (Abcam).

### Example 8

Immunoturbidimetry assay for determination of vitamin D status with soluble megalin. Ni-NTA purified soluble megalin M1 or M2 protein fragment (3 μg) is first mixed with human serum or plasma samples (30 μl). Samples are incubated with 50 μl of pre-equilibrated Ni-NTA resin at 4° C. overnight. The resin is washed 3 times with H-buffer+20 mM imidazole. Bound complex is eluted with H-buffer containing 200 mM imidazole. The eluted complex is contacted in aqueous solution with an antibody against vitamin D binding protein. The increase in turbidity is measured with a standard turbidimeter and compared with standard values of known 25-hydroxvitamin D₃ concentration.

Alternatively, nanoparticles, for example, latex nanoparticles (aprox. 150 nm), are coated with megalin M1 or M2 fragment and incubated with a serum or plasma sample. The increase in turbidity is then measured and compared to standard values of known 25-hydroxvitamin D3 concentration.

### Example 9

ELISA binding assay for linearity range determination. A microtiter plate was coated with purified megalin M1 or M2 protein fragment (1 μg/100 μl diluted in PBS1X) by incubation at RT for 2 h. Mixtures of DBP and 25-hydroxvitamin D₃ (VD₃), 25-hydroxvitamin D₂ (VD₂) or 24,25-hydroxvitamin D (24,25VD) were prepared by mixing 20 μg DBP and serial dilution of VD (0, 0.78, 1,56, 3,125, 6.25, 12.5, 25, 50 ng) in 100 μl PBS. The mixture was incubated at 37° C. for 1 h. After plate blocking, the above prepared DBP-VD mixture was added to megalin-coated wells and incubated at 4° C., overnight. Then, the plate was incubated with 100 μl diluted rabbit-anti-DBP antibody (1:1000 diluted in blocking buffer) at RT for 2 h, followed by incubation with 100 μl of HRP-conjugated donkey anti-rabbit antibody (1:500 diluted in blocking buffer) at 37° C., 1 h. After substrate reaction, the absorbance was read at 450 nm. The assay was linear up to 50 ng/ml. The sensitivity limit was determined to be 2.0 ng/ml.

### Example 10

Microscale Thermophoresis assay. Purified DBP (Merck, 345802) was labeled with the red fluorescent dye NT-647 by using a Monolith Protein Labeling Kit Red (NanoTemper Technologies, Munich, DE). Ni-NTA-purified megalin fragments (M1, M2) were titrated in the range of 0.488 to 1000 nmol/L. Purified DBP (Merck, 345802) was likewise labeled with the red fluorescent dye NT-647 by using the Monolith Protein Labeling Kit Red. 25-hydroxvitamin D₃ (VD₃) was titrated in concentrations in the range of 0.0488 to 100 nmol/L. 25-hydroxvitamin D₂ (VD₂), 24,25-hydroxvitamin D (24,25VD) and 3epi25(OH)VD₃ were titrated in concentrations in the range of 0.0163 to 37 nmol/L. 37.8 nM of VD₃, VD₂ and 24,25VD was respectively added to 50 nM of NT-647 labeled DBP. A Monolith NT.115 device (NanoTemper Technologies) was used for measurements. NT Analysis software version 1.427 (NanoTemper Technologies) was used for analysis. Parameters: laser power, 100%; LED, 80; laser on-time, 30 seconds; laser off-time, 5 seconds; temperature, 25° C. FNorm: normalized fluorescence; Kd: dissociation constant.

The dissociation constants Kd of the vitamin D metabolites to DBP gave the following ranking of the binding affinities. 3epi25(OH)₂VD3>25(OH)₂VD3≥25(OH)₂VD₂>24,25(OH)₂VD3>1,25(OH)₂VD3. The binding affinity of DBP-VD₂ is comparable to Kd of DBP-VD₃. The binding of 24,25(OH)VD to DBP is marginally lower. The high binding affinity of the 3C epimer of 25(OH)D₃ is surprising and will require further investigation as this epimer seems, therefore, particularly useful as a food supplement, if not toxic for other reasons.

### Example 11

Epitopes specific for the ternary complex of 25OH-vitD₃:DBP:megalin-5-peptide.

The Immune Epitope Database and Analysis Resource (IEDB) is a freely available online resource for epitope prediction and analysis. It provides tools and databases for researchers to analyze and predict immunologically relevant epitopes. The ternary complex (also named by us holoprotein DBP:25OHD3″) was searched in conjunction with the IEDB using the ElliPro tool (http://tools.immuneepitope.org/ellipro/result/predict), which predicts specific antigenic epitopes based on solvent accessibility and flexibility. This search resulted in the prediction of eight linear epitopes and two discontinuous antigenic epitopes on the DBP sequence of the ternary complex which were not present on isolated DBP. Further analysis led to the following discontinuous antigenic epitopes within chain A of DBP that could be specific for the said ternary complex alone:

(TH-17 aa)
SEQ ID NO: 11
T-A-E-C-C-T-K-R-K-L-C-M-A-A-L-K-H
(EL-12 aa)
SEQ ID NO: 12 (12 aa)
E-S-A-S-E-D-C-M-A-K-E-L-

The two peptides, TH-17 and EL-12 were both synthesized chemically by ProteoGenix, Schiltigheim, FR, yielding approximately some hundreds milligrams each. Following synthesis, these peptides underwent conjugation to KLH (keyhole limpet hemocyanin) at their N-termini. A combination of the conjugated peptides was then used to immunize two rabbits over a 70-day period, including booster immunizations. The resulting outcomes included 0.5 to 1 ml of pre-immune serum (prior immunization), 2 to 20 mg of purified antibodies, as well as some ten milligrams of purified peptides TH-17 and EL-12 for subsequent comparative experiments. Subsequently, the entire serum from both animals (rabbits) was purified against each peptide separately to yield specific polyclonal antibodies targeting the artificial peptides, aimed at capturing the discontinuous epitopes of the ternary complex (25OH-vitD₃:DBP:megalin peptide).

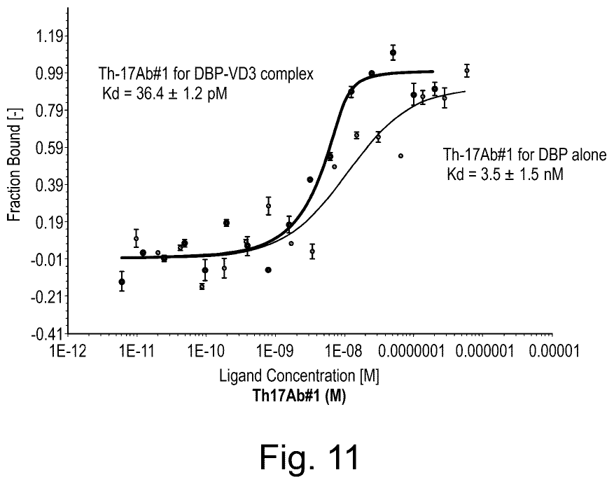

MicroScale Thermophoresis (MST) is the method of choice for biophysically analyzing the binding affinity of the purified polyclonal anti-TH17-antibodies. The targets are their interactions with the ternary complex (25OH-vitD₃:DBP:megalin peptide) and the apoprotein form of DBP (non-complexed DBP). MST is the tool for directly measuring antibody affinities in solution, bypassing the need for surface immobilization. The comparative results of the MST binding affinity analysis for the purified polyclonal rabbit anti-TH17 antibodies are presented in the diagram shown in FIG. 11.

Notably, anti-TH17-antibodies obtained from rabbit #1 had a significantly higher binding affinity for the holistic megalin-bound complex with DBP:25OH-vitamin-D₃ (VD3) (Kd=36.4 pM) compared to the apoprotein DBP alone (Kd=3.5±1.5 nM). Pre-immune sera from both animals showed “no” or negligible binding to the megalin-bound DBP:VD3 complex (estimated >1033 nM±1.5 nM for animal #1 and estimated >1025±0.9 nM for animal #2). In addition, the purified polyclonal anti-TH17-antibodies from animal #2 exhibited a binding affinity (Kd) as measured by MST of 9.67±1.5 nM for the megalin-bound DBP-VD3 complex, while exhibiting minimal or weak binding to the apoprotein DBP.

The definition of an antigenic epitope specific for the megalin-bound complex with DBP:25OH-vitamin D₃ (VD3) and the generation of specific antibodies against megalin-bound DBP:VD3 thus allow the construction of homogeneous as well as heterogeneous immunoassays for the determination of the endocytable vitamin D status, as this is equivalent to the concentration of the ternary complex, which physiologically must be present as a holoprotein formed by DBP, 25-OH-vitamin D₃ (VD3) and megalin and/or cubilin. In a heterogenous ELISA, the megalin or cubilin peptide or fragment can be immobilized on the stationary phase (microtitre plate) where it then binds the DBP:VD3 complex in the test fluid. The amount of megalin-bound complex of DBP:VD3 (25-Hydroxy-vitamin D₃) can be determined directly using enzyme-labelled anti-TH17 antibodies as described.

1. A method for adaptation of vitamin D supplementation therapy in a subject, by measuring vitamin D and its metabolites in a sample of bodily fluid from the subject, which sample may contain endogenous vitamin D binding protein (DBP), comprising the steps of:
a) contacting said sample with megalin and/or a fragment thereof under binding conditions to form a ternary complex comprising DBP, vitamin D metabolite and megalin or a fragment thereof;
b) determining an amount of DBP bound by megalin or a fragment thereof; and
c) relating the determined amount of megalin-bound DBP to an effective vitamin D status of said subject, wherein a higher amount of megalin-bound DBP indicates a higher amount of activatable circulatory vitamin D metabolites, and
d) adapting a vitamin D supplementation therapy to the effective vitamin D status of the subject.

2. The method according to claim 1, wherein said fragment of megalin comprises amino acid sequences of SEQ ID NO:01 through SEQ ID NO:10.

3. Method according to claim 1, wherein said fragment of megalin comprises amino acid SEQ ID NO:01 or SEQ ID NO:2 or combinations thereof.

4. The method of claim 1, wherein the fragment of megalin binds exclusively vitamin D binding protein (DBP) and no other ligands of megalin.

5. The method of claim 1 comprising a determining of the bound vitamin D metabolite.

6. The method of claim 1, comprising a differential measurement of 25-hydroxy-vitamin D₃ in the presence of 25-hydroxyvitamin D₂, 24,25-dihydroxyvitamin D₂, or 24,25-dihydroxyvitamin D₃.

7. Method according to claim 1, comprising the addition of a known amount of vitamin D binding protein to the sample.

8. Method according to claim 1, further contacting said sample with cubilin and/or a soluble fragment thereof.

9. Method according to any claim 1, wherein said complex with DBP is determined by particle-enhanced immunoturbidimetry or nephelometry.

10. Method according to claim 1, wherein said complex with DBP is determined by an immunoassay selected from the group ELISA (enzyme-linked immunosorbent assay), RIA (radioimmunoassay), FIA (fluorescence immunoassay), L1A (luminescence immunoassay), or ILMA.

11. Method according to claim 1, comprising the steps of:
(a) providing a defined amount of megalin and/or a soluble fragment thereof coupled to a solid phase;
(b) contacting the sample with the solid phase having coupled megalin and/or a soluble fragment thereof;
(c) creating conditions to allow binding of megalin and/or a soluble fragment thereof to vitamin D binding protein (DBP) and vitamin D metabolites, wherein vitamin D binding protein (DBP) alone does not bind to megalin and/or a soluble fragment thereof; and washing the solid phase;
(d) providing an antibody complex recognizing the complex comprising DBP, vitamin D or its metabolites and megalin or a soluble fragment thereof;
(e) contacting said megalin and/or a soluble fragment thereof bound to vitamin D binding protein (DBP) and vitamin D metabolites with the antibody recognizing the complex comprising DBP, vitamin D or its metabolites and megalin or a soluble fragment thereof, and immobilizing the complex on the solid phase; and
(f) determining the amount of antibody bound to the solid phase, and quantitating the vitamin D metabolites in the bodily fluid, wherein a higher amount of antibody bound to the solid phase indicates a higher amount of activatable vitamin D metabolites.

12. Test kit for use in a method of determining a vitamin D status in a sample of bodily fluid, comprising an antibody against DBP and a fragment or amino acid sequence of megalin, optionally coupled to a carrier.

13. Composition for measuring vitamin D metabolites in a sample of bodily fluid, comprising antibodies against an epitope of a DBP and a fragment of megalin.


SEQUENCE LISTING (12 sequences)

SEQ ID NO: 1 - protein, 15 aa, Homo sapiens
DNGNCIHRAWLCDRD

SEQ ID NO: 2 - protein, 15 aa, Homo sapiens
GCTHECVQEPFGAKC

SEQ ID NO: 3 - protein, 5 aa, Homo sapiens
CVQEP

SEQ ID NO: 4 - protein, 5 aa, Homo sapiens
IHRAW

SEQ ID NO: 5 - protein, 15 aa, Homo sapiens
SDFNGGCTHECVQEP

SEQ ID NO: 6 - protein, 15 aa, Homo sapiens
CYNMRGSFRCSCDTG

SEQ ID NO: 7 - protein, 15 aa, Homo sapiens
FSFPCKNGRCVPNYY

SEQ ID NO: 8 - protein, 346 aa, Homo sapiens
IPADWRCDGTKDCSDDADEIGCAVVTCQQGYFKCQSEGQCIPNSWVCDQDQDCDDGSDERQDCSQSTCSSHQITCSNGQCIPSEYRCDHVRDCPDGADENDCQYPTCEQLTCDNGACYNTSQKCDWKVDCRDSSDEINCTEICLHNEFSCGNGECIPRAYVCDHDNDCQDGSDEHACNYPTCGGYQFTCPSGRCIYQNWVCDGEDDCKDNGDEDGCESGPHDVHKCSPREWSCPESGRCISIYKVCDGILDCPGREDENNTSTGKYCSMTLCSALNCQYQCHETPYGGACFCPPGYIINHNDSRTCVEFDDCQIWGICDQKCESRPGRHLCHCEEGYILERGQYCK

SEQ ID NO: 9 - protein, 406 aa, Homo sapiens
EQCGLFSFPCKNGRCVPNYYLCDGVDDCHDNSDEQLCGTLNNTCSSSAFTCGHGECIPAHWRCDKRNDCVDGSDEHNCPTHAPASCLDTQYTCDNHQCISKNWVCDTDNDCGDGSDEKNCNSTETCQPSQFNCPNHRCIDLSFVCDGDKDCVDGSDEVGCVLNCTASQFKCASGDKCIGVTNRCDGVFDCSDNSDEAGCPTRPPGMCHSDEFQCQEDGICIPNFWECDGHPDCLYGSDEHNACVPKTCPSSYFHCDNGNCIHRAWLCDRDNDCGDMSDEKDCPTQPFRCPSWQWQCLGHNICVNLSVVCDGIFDCPNGTDESPLCNGNSCSDFNGGCTHECVQEPFGAKCLCPLGFLLANDSKTCEDIDECDILGSCSQHCYNMRGSFRCSCDTGYMLESDGRTCK

SEQ ID NO: 10 - protein, 495 aa, Homo sapiens
ERCGASSFTCSNGRCISEEWKCDNDNDCGDGSDEMESVCALHTCSPTAFTCANGRCVQYSYRCDYYNDCGDGSDEAGCLFRDCNATTEFMCNNRRCIPREFICNGVDNCHDNNTSDEKNCPDRTCQSGYTKCHNSNICIPRVYLCDGDNDCGDNSDENPTYCTTHTCSSSEFQCASGRCIPQHWYCDQETDCFDASDEPASCGHSERTCLADEFKCDGGRCIPSEWICDGDNDCGDMSDEDKRHQCQNQNCSDSEFLCVNDRPPDRRCIPQSWVCDGDVDCTDGYDENQNCTRRTCSENEFTCGYGLCIPKIFRCDRHNDCGDYSDERGCLYQTCQQNQFTCQNGRCISKTFVCDEDNDCGDGSDELMHLCHTPEPTCPPHEFKCDNGRCIEMMKLCNHLDDCLDNSDEKGCGINECHDPSISGCDHNCTDTLTSFYCSCRPGYKLMSDKRTCVDIDECTEMPFVCSQKCENVIGSYICKCAPGYLREPDGKTCR

SEQ ID NO: 11 - protein, 17 aa, synthetic construct
TAECCTKRKLCMAALKH

SEQ ID NO: 12 - protein, 12 aa, synthetic construct
ESASEDCMAKEL